(19) United States
(12) Patent Application Publication
(10) Pub. No.: US 2022/0267764 A1
(43) Pub. Date: Aug. 25, 2022

(54) METHODS AND SYSTEMS FOR RNA-SEQ PROFILING

(71) Applicant: Honeycomb Biotechnologies, Inc., Weston, MA (US)

(72) Inventor: Todd Gierahn, Brookline, MA (US)

(21) Appl. No.: 17/681,060

(22) Filed: Feb. 25, 2022

Related U.S. Application Data
(63) Continuation of application No. PCT/US2020/049558, filed on Sep. 4, 2020.
(60) Provisional application No. 62/897,003, filed on Sep. 6, 2019.

(51) Int. Cl. C12N 15/10; C12Q 1/6855; C12Q 1/6806; C12Q 1/6874

(52) U.S. Cl. CPC C12N 15/1096; C12Q 1/6855; C12N 15/1065; C12Q 1/6874; C12Q 1/6806

(57) ABSTRACT

Disclosed herein are methods for counting nucleic acid molecules (e.g., RNA molecules) of a sample by randomly truncating the nucleic acid molecules at a truncation base position within the nucleic acid molecules to produce truncated nucleic acid molecules, amplifying and sequencing the truncated nucleic acid molecules to produce sequencing reads, aligning the sequencing reads to a reference sequence to produce aligned sequencing reads, and identifying a number of nucleic acid molecules using truncation locations of aligned sequencing reads. Also disclosed herein are methods for constructing sequencing libraries that preserve truncation positions of the nucleic acid molecules. Also disclosed herein are methods for depleting or enriching a sample for one or more target sequences, using sets of blocking oligonucleotides corresponding to the one or more target sequences.

### CROSS-REFERENCE OF RELATED APPLICATIONS

This application is a continuation of International Application No. PCT/US2020/049558, filed Sep. 4, 2020 which claims benefit of U.S. Provisional Patent Application No. 62/897,003, filed Sep. 6, 2019, which is incorporated herein by reference in its entirety.

### SEQUENCE LISTING

The instant application contains a Sequence Listing which has been submitted electronically in ASCII format is hereby incorporated by reference in its entirety. Said ASCII copy, created on Feb. 25, 2022, is named 52946_703_301_SL.txt and is 2,522 bytes in size.

### BACKGROUND OF THE INVENTION

RNA-seq has become a mainstay technique for measuring the expression of genes in a sample including down to a single cell. Several high throughput approaches have been developed for single cell RNA-seq analysis. Most revolve around the addition of a unique barcode to the 3′ end of all transcripts derived from a single cell during reverse transcription. So-called 3′-barcoded libraries are typically amplified, fragmented into proper sequencing library size, and then attached to adaptor sequences for sequencing on commercial platforms. The sequencing reads are then grouped by barcode to identify the transcripts captured from each original cell. Critical for any manipulation of these libraries is the maintenance of the link between the 3′ barcode and the transcript sequence, otherwise the cellular origin of a given transcript is lost.

### SUMMARY

In an aspect, described herein is a method for counting nucleic acid molecules of a sample, comprising: (a) obtaining a sample comprising a plurality of template nucleic acid molecules; (b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules; (c) amplifying at least a portion of said plurality of truncated nucleic acid molecules to produce a plurality of amplified nucleic acid molecules, wherein said truncation base positions are preserved in said amplified nucleic acid molecules; (d) sequencing at least a portion of said plurality of amplified nucleic acid molecules to produce a plurality of sequencing reads, wherein each of said plurality of sequencing reads comprises a truncation location corresponding to said truncation base position of said corresponding amplified nucleic acid molecule; (e) aligning at least a portion of said plurality of sequencing reads to a reference sequence, thereby producing a plurality of aligned sequencing reads; and (f) identifying a number of template nucleic acid molecules present in said sample using truncation locations of said plurality of aligned sequencing reads. In some embodiments, the truncating comprises cleaving said plurality of template nucleic acid molecules. In some embodiments, the truncating comprises performing base-catalyzed hydrolysis, ultrasonic shearing, or partial enzymatic degradation, of said plurality of template nucleic acid molecules. In some embodiments, the truncating comprises making a copy of at least a portion of said plurality of template nucleic acid molecules. In an aspect, method for counting nucleic acid molecules of a sample, comprising: (a) obtaining a sample comprising a plurality of template nucleic acid molecules; (b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules; (c) amplifying a portion of said plurality of truncated nucleic acid molecules to produce a plurality of amplified nucleic acid molecules, wherein said truncation base positions are preserved in said amplified nucleic acid molecules; (d) sequencing a portion of said plurality of amplified nucleic acid molecules to produce a plurality of sequencing reads, wherein each of said plurality of sequencing reads comprises a truncation location corresponding to said truncation base position of said corresponding amplified nucleic acid molecule; (e) aligning a portion of said plurality of sequencing reads to a reference sequence, thereby producing a plurality of aligned sequencing reads; and (f) identifying a number of template nucleic acid molecules present in said sample using truncation locations of said plurality of aligned sequencing reads. In one aspect, described herein is a method for counting nucleic acid molecules of a sample, comprising: (a) obtaining a sample comprising a plurality of template nucleic acid molecules; (b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule and making a copy of at least a portion of said template nucleic acid molecules, thereby producing a plurality of truncated nucleic acid molecules; (c) amplifying at least a portion of said plurality of truncated nucleic acid molecules to produce a plurality of amplified nucleic acid molecules, wherein said truncation base positions are preserved in said plurality of amplified nucleic acid molecules; (d) sequencing at least a portion of said amplified nucleic acid molecules to determine a number of unique truncation base positions present in said at least a portion of said amplified nucleic acid molecules; and (e) identifying a number of template nucleic acid molecules present in said sample using said number of unique truncation base positions. In one aspect, described herein is a method for counting nucleic acid molecules of a sample, comprising: (a) obtaining a sample comprising a plurality of template nucleic acid molecules; (b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule and making a copy of a portion of said template nucleic acid molecules, thereby producing a plurality of truncated nucleic acid molecules; (c) amplifying a portion of said plurality of truncated nucleic acid molecules to produce a plurality of amplified nucleic acid molecules, wherein said truncation base positions are preserved in said plurality of amplified nucleic acid molecules; (d) sequencing a portion of said amplified nucleic acid molecules to determine a number of unique truncation base positions present in said a portion of said amplified nucleic acid molecules; and (e) identifying a number of template nucleic acid molecules present in said sample using said number of unique truncation base positions. In some embodiments, the method comprises aligning at least a portion of said plurality of sequencing reads to a reference sequence, thereby producing a plurality of aligned sequencing reads. In some embodiments, the method comprises processing at least a portion of said amplified nucleic acid molecules to produce a sequencing library, wherein said truncation base positions are preserved in said sequencing library. In some embodiments, the plurality of template nucleic acid molecules comprises deoxyribonucleic acid (DNA) molecules. In some embodiments, the plurality of template nucleic acid molecules comprises complementary DNA (cDNA) molecules. In some embodiments, the plurality of template nucleic acid molecules comprises ribonucleic acid (RNA) molecules. In some embodiments, said sample comprises one or more barcoded beads, and wherein said template nucleic acid molecules are cDNA molecules attached to said barcoded beads. In some embodiments, said cDNA molecules are obtained by reverse transcription of RNA molecules that are released from cellular single cell samples. In some embodiments, the truncating comprises making said copy of said template nucleic acid molecules from said truncation base position. In some embodiments, the truncating comprises making said copy of said template nucleic acid molecules, wherein said truncation base position is preserved in said copy. In some embodiments, said truncating comprises forming a plurality of second strand cDNA molecules from said plurality of template nucleic acid molecules, wherein said truncation base positions are preserved in said plurality of second strand cDNA molecules. In some embodiments, the truncating comprises forming a plurality of second strand cDNA molecules from said plurality of template nucleic acid molecules, wherein said plurality of second strand cDNA molecules comprises said truncation base positions. In some embodiments, the method comprises contacting said plurality of template nucleic acid molecules with a plurality of second strand primers, wherein each of said plurality of second strand primers comprises a 5′ universal primer sequence and a 3′ sequence complementary to a sequence of said template nucleic acid molecules, and wherein said 3′ sequence comprises a random sequence. In some embodiments, the method comprises extending said plurality of second strand primers to produce said plurality of second strand cDNA molecules. In some embodiments, the method comprises performing random transposon insertion of said plurality of second strand cDNA molecules to randomly fragment said plurality of second strand cDNA molecules. In some embodiments, the 3′ sequence comprises 5, 6, 7, 8, 9, 10, 11, 12, 13, 14, 15, or 16 bases. In some embodiments, the 3′ sequence comprises 9 or 10 bases. In some embodiments, the 3′ sequence is linked on its 5′ side to said universal primer. In some embodiments, the second strand primers comprise a sided sequence (e.g., 5′ SS). In some embodiments, the SS comprises 2 to 5 bases. In some embodiments, the SS comprises 5 to 9 bases. In some embodiments, the SS flanks said universal primer sequence. In some embodiments, said SS flanks said universal primer sequence and said 3′ sequence. In some embodiments, said template nucleic acid molecules comprise, in 5′ to 3′ direction, a universal primer sequence, a sided sequence (SS), a sample barcode, a poly(dT) sequence, and a sequence that is complementary to a sequence of a target nucleic acid. In some embodiments, the template nucleic acid molecules comprise a sided sequence (e.g., 3′ SS). In some embodiments, the 3′ SS comprises 2 to 5 bases. In some embodiments, the 3′ SS comprises 5 to 7 bases. In some embodiments, each of said SS independently comprises a known sequence. The SS can be a designed sequence. In some embodiments, the 3′ SS flanks said universal primer sequence. In some embodiments, said obtaining comprises generating said template nucleic acid molecules by performing reverse transcription of a plurality of target nucleic acid molecules released from one or more cellular samples. In some embodiments, the method comprises performing reverse transcription of a plurality of target nucleic acid molecules to generate a plurality of template nucleic acid molecules. In some embodiments, the method comprises partitioning said one or more cellular samples across a plurality of phase partitions such that an individual cell is captured in single partition. In some embodiments, the method comprises partitioning said plurality of target nucleic acid molecules across a plurality of phase partitions. In some embodiments, the method comprises releasing said target nucleic acid molecules from said single cell, capturing said target nucleic acid molecules from a single cell onto a barcoded bead, generating template nucleic acid molecules by performing reverse transcription of said target nucleic acid molecules and optionally pooling said plurality of template nucleic acid molecules across said plurality of phase partitions. In some embodiments, the method comprises pooling said plurality of template nucleic acid molecules across said plurality of phase partitions. In some embodiments, the plurality of phase partitions comprises microwells or droplets. In some embodiments, the method comprises tagging each of said plurality of target nucleic acid molecules with a unique sample barcode among a plurality of sample barcodes, each of said plurality of sample barcodes comprising a set of one or more nucleotide bases. In some embodiments, the method comprises tagging each of said plurality of target nucleic acid molecules with a sample barcode that is indicative of a sample with which said target nucleic acid molecules are associated. In some embodiments, the sample barcode is identical among all of said plurality of target nucleic acid molecules in said sample. In some embodiments, the method comprises releasing said plurality of target nucleic acid molecules from said one or more cellular sample. In some embodiments, the method comprises using a plurality of chain-terminating nucleotides to perform said random truncation at said truncation base position. In some embodiments, the plurality of chain-terminating nucleotides comprises dideoxynucleotides. In some embodiments, the plurality of chain-terminating nucleotides is configured to produce a truncation size distribution among said plurality of truncated nucleic acid molecules. In some embodiments, the method comprises chemically labeling a 3′ carbon position of each of said plurality of chain-terminating nucleotides to enable chemical ligation of a universal 5′ primer site of said at least said portion of said plurality of template nucleic acid molecules. In some embodiments, the truncated nucleic acid molecules are amplified using polymerase chain reaction (PCR) amplification. In some embodiments, the PCR amplification comprises suppression PCR amplification. In some embodiments, the method comprises a second PCR amplification, during which the truncation sites are preserved. In some embodiments, the method comprises a second PCR amplification that re-establishes directionality of said sequencing library. In some embodiments, the sequencing library comprises known sided sequences (SS) on a 3′ and a 5′ side of nucleic acid molecules of said sequencing library. In some embodiments, the 3′ and 5′ SS defines the 3′ and 5′ direction of the sequencing library respectively. In some embodiments, said 3′ SS is a copy of the SS in the template nucleic acid molecules, and said 5′ SS is a copy of the SS in the second strand primer. In some embodiments, the 3′ SS is common to all the nucleic acid molecules of the library. In some embodiments, the 5′ SS is common to all the nucleic acid molecules of the library. The SS can also be unique. In some embodiments, the sided sequences have a length of 2 to 5 bases. In some embodiments, the sided sequences have a length of 5 to 9 bases. In some embodiments, the sided sequences have a length of about 5 bases. In some embodiments, the sided sequences have a length of about 6 bases. In some embodiments, the sided sequences have a length of about 7 bases. In some embodiments, the sided sequences have a length of about 8 bases. In some embodiments, the sided sequences have a length of about 9 bases. In some embodiments, the sided sequences have a length of 5 to 12 bases. In some embodiments, the second PCR amplification comprises amplifying suppression PCR products with indexing primers, wherein said indexing primers comprise, in a 5′-3′ direction, an adaptor sequence, an index sequence for indexing of said sequencing library, and a custom sequencing primer sequence. In some embodiments, the custom sequencing primer sequence comprises a sequence complementary to a portion of a UPS sequence and to a sided sequence. In some embodiments, the sided sequence defines a 3′ or a 5′ side of said sequencing library. In some embodiments, said index primers comprise sequences that are specific for the 5′ and 3′ sided sequence with 5′ tails containing the appropriate adaptor. In some embodiments, the custom sequencing primer sequence has a length of about 25-40 nucleotides. In some embodiments, the second PCR amplification comprises using a PCR annealing time of about 5 minutes. In some embodiments, the second PCR amplification is performed without purification of suppression PCR products of said suppression PCR amplification. In some embodiments, the method comprises correlating a number of said plurality of template nucleic acid molecules, based at least in part on determining a quantitative measure of said plurality of aligned sequencing reads having a same mapping base location. In some embodiments, the method comprises identifying said number of template nucleic acid molecules present in said sample using a number of said plurality of aligned sequencing reads having a same mapping base location, and a same sample index. In some embodiments, the method comprises, prior to (c), tagging each of said plurality of truncated nucleic acid molecules with a non-unique barcode among a plurality of non-unique barcodes, each of said plurality of non-unique barcodes comprising a set of one or more nucleotide bases. In some embodiments, each of said plurality of non-unique barcodes comprises a set of from about 2 to about 100 nucleotide bases, from about 2 to about 50 nucleotide bases, from about 2 to about 20 nucleotide bases, or from about 2 to about 10 nucleotide bases. In some embodiments, the method comprises correlating a number of said plurality of template nucleic acid molecules, based at least in part on determining a quantitative measure of said plurality of aligned sequencing reads having a same mapping base location and a same non-unique barcode. In some embodiments, each of said plurality of template nucleic acid molecules comprises a unique sample barcode among a plurality of sample barcodes. In some embodiments, each of said plurality of sample barcodes comprises a set of about 5 to about 100 nucleotide bases. In some embodiments, the method comprises identifying said number of template nucleic acid molecules present in said sample using a number of said plurality of aligned sequencing reads having a same mapping base location, a same non-unique barcode, and a same sample index. In some embodiments, the method comprises, prior to (c), tagging each of said plurality of truncated nucleic acid molecules with a unique molecular identifier (UMI) among a plurality of UMIs, each of said plurality of UMIs comprising a set of one or more nucleotide bases. In some embodiments, each of said plurality of UMIs comprises a set of about 5 to about 100 nucleotide bases. In some embodiments, the method comprises correlating a number of said plurality of template nucleic acid molecules, based at least in part on determining a quantitative measure of said plurality of aligned sequencing reads having a same mapping base location and a same UMI. In some embodiments, each of said plurality of template nucleic acid molecules comprises a unique sample barcode among a plurality of sample barcodes. In some embodiments, each of said plurality of sample barcodes comprises a set of about 5 to about 100 nucleotide bases. In some embodiments, the method comprises identifying said number of template nucleic acid molecules present in said sample using a number of said plurality of aligned sequencing reads having a same mapping base location, a same UMI, and a same sample index. In some embodiments, each of said template nucleic acid molecules comprises a common sample barcode. In some embodiments, the method comprises enriching or depleting said plurality of amplified nucleic acid molecules for one or more target sequences. In some embodiments, the method comprises depleting said plurality of amplified nucleic acid molecules for one or more target sequences. In some embodiments, the one or more target sequences comprise ribosomal RNA (rRNA) sequences. In some embodiments, the method comprises using one or more blocking oligonucleotides, wherein each of said one or more blocking nucleotides comprises a target sequence of said one or more target sequences. In some embodiments, the method comprises using one or more blocking oligonucleotides, wherein each of said one or more blocking nucleotides comprises a copy of a target sequence of said one or more target sequences, or a fragment thereof. In some embodiments, the method comprises enriching said plurality of amplified nucleic acid molecules for one or more target sequences. In some embodiments, the one or more target sequences comprise a variable region in a T-cell or B-cell receptor, a single nucleotide polymorphism (SNP), a splicing junction, or a combination thereof. In some embodiments, the sequencing comprises whole genome sequencing (WGS). In some embodiments, the sequencing comprises massively parallel sequencing. In some embodiments, the sequencing performed at a depth of no more than about 20×. In some embodiments, the sequencing comprises obtaining a first sequencing read and a second sequencing read. In some embodiments, the sample barcode is captured in said first sequencing read. In some embodiments, the truncation location corresponding to said truncation base position is captured in said second read. In some embodiments, the template nucleic acid molecules are aligned to said reference sequence according to said second read. In some embodiments, the non-unique barcodes are captured in said second sequencing read. In some embodiments, the second read comprises sequencing from about 10 to about 50 bases in said template nucleic acid molecules. In some embodiments, obtaining said first sequencing read comprises sequencing a 3′ side sequence of said template nucleic acid and obtaining said second sequencing read comprises sequencing a 5′ side sequence of said template nucleic acid. In some embodiments, the sample is a biological sample. In some embodiments, the truncating is performed without performing a tagmentation step. In some embodiments, the method comprises adjusting said number of template nucleic acid molecules identified as present in said sample, wherein said adjusting comprises calculating a maximum likelihood estimate of a number of said template nucleic acid molecules that have a same truncation base position. In some embodiments, the maximum likelihood estimate is calculated using a Poisson statistical distribution.

In one aspect, disclosed herein is a method for depleting a sample for one or more target sequences, comprising: (a) obtaining a sample comprising a plurality of template nucleic acid molecules, wherein said template nucleic acid molecules comprise one or more target sequences; (b) combining said plurality of template nucleic acid molecules with a set of blocking oligonucleotides, wherein said set of blocking oligonucleotides is configured to bind with at least one of said one or more target sequences, thereby annealing at least one of said one or more target sequences with at least one of said set of blocking oligonucleotides; (c) contacting said plurality of template nucleic acid molecules with a plurality of second strand primers, wherein said plurality of second strand primers comprises a 5′ universal primer sequence and a 3′ sequence complementary to a sequence of said template nucleic acid molecules; and (d) extending said plurality of second strand primers to produce a plurality of second strand nucleic acid molecules, thereby depleting at least one of said one or more target sequences. In some embodiments, the one or more target sequences comprise ribosomal RNA (rRNA) sequences, sequences of variable regions in T-cell and B-cell receptors, single nucleotide polymorphism (SNP) sequences, splicing junction sequences, or a combination thereof. In some embodiments, the set of blocking oligonucleotides is sufficient to cover an entire sequence of one or more of said one or more target sequences. In some embodiments, each of said set of blocking oligonucleotides comprises between about 20 to about 100 bases. In some embodiments, the 3′ sequence has a first annealing temperature, wherein said set of blocking oligonucleotides has a second annealing temperature greater than said first annealing temperature, and wherein said method further comprises performing (c) at a third annealing temperature greater than said first annealing temperature and less than said second annealing temperature.

In one aspect, disclosed herein is a method for enriching a sample for one or more target sequences, comprising: (a) obtaining a sample comprising a plurality of template nucleic acid molecules, wherein said template nucleic acid molecules comprise one or more target sequences; (b) combining said plurality of template nucleic acid molecules with a set of blocking oligonucleotides, wherein said set of blocking oligonucleotides comprises a sequence complementary to a template nucleic sequence that is 3′ to one of said target sequences, thereby annealing said template nucleic acid sequence that is 3′ to one of said target sequences with at least one of said set of blocking oligonucleotides; (c) contacting said plurality of template nucleic acid molecules with a plurality of second strand primers, wherein said plurality of second strand primers comprises a 5′ universal primer sequence and a 3′ sequence complementary to a sequence of said template nucleic acid; and (d) extending said second strand primers to produce a plurality of second strand nucleic acid molecules, thereby enriching at least one of said one or more target sequences. In some embodiments, the method further comprises extending said second strand nucleic acid molecules through a region of said second strand cDNA molecule corresponding to a blocking oligonucleotide of said set of blocking oligonucleotides to acquire a 3′ barcode and a 3′ UPS sequence. In some embodiments, the method further comprises performing a two-step extension reaction using a mesophilic DNA polymerase and a thermophilic DNA polymerase. In some embodiments, performing said two-step extension reaction comprises initiating extension at a first temperature less than an extension temperature of said set of blocking oligonucleotides to extend said 3′ sequences, and continuing extension at a second temperature greater than said extension temperature of said set of blocking oligonucleotides, to dissociate said set of blocking oligonucleotides from said plurality of second strand nucleic acid molecules. In some embodiments, the method further comprises using a polymerase with high strand displacement activity in said second strand synthesis reaction to displace said set of blocking oligonucleotides. In some embodiments, the method further comprises annealing said set of blocking oligonucleotides and said 3′ sequences. In some embodiments, the method further comprises extending said set of blocking oligonucleotides using a DNA polymerase and one or more cleaving enzymes corresponding to said set of blocking oligonucleotides. In some embodiments, the method further comprises cleaving said set of blocking oligonucleotides using one or more cleaving enzymes corresponding to said set of blocking oligonucleotides, and extending said set of blocking oligonucleotides using a DNA polymerase. In some embodiments, the 3′ sequence complementary to a sequence of said template nucleic acid comprises a random sequence. In some embodiments, the 3′ sequence complementary to a sequence of said template nucleic acid is complementary to a template nucleic sequence 5′ to one of said target sequences. In some embodiments, each of said set of blocking oligonucleotides comprise at most 100, at most 75, at most 50, at most 40, at most 30, at most 25, at most 20, at most 15, at most 10, or at most 5 bases. In some embodiments, each of said set of blocking oligonucleotides comprise at least 2, at least 3, at least 4, at least 5, at least 10, at least 15, at least 20, at least 25, at least 30, at least 40, at least 50, or at least 75 bases.

In one aspect, disclosed herein is a method for constructing a sequence library for sequencing a plurality of template nucleic acid molecules, comprising: contacting a plurality of template nucleic acid molecules with a plurality of second strand primers, wherein each of said plurality of second strand primers comprises a 5′ universal primer sequence and a 3′ sequence complementary to a sequence of said template nucleic acid molecules; extending said plurality of second strand primers to produce a plurality of second strand nucleic acid molecules; and amplifying said plurality of second strand nucleic acid molecules from (b) with a plurality of indexing primers, wherein said plurality of indexing primers comprise, in a 5′-3′ direction, an adaptor sequence, an index sequence for indexing of said sequencing library, and a custom sequencing primer sequence. In some embodiments, said 3′ sequence hybridizes with said template nucleic acid molecules in a site-nonspecific fashion. In some embodiments, said 3′ sequence comprises a random sequence.

In one aspect, disclosed herein is a system comprising (a) a plurality of beads; (b) a plurality of cDNA molecules, wherein each of said cDNA molecules is attached to one of said beads, wherein said plurality of cDNA molecules each comprises a sample barcode, a sided sequence, and a universal primer sequence; and (c) a plurality of second strand primers for performing second strand synthesis of said plurality of cDNA molecules to produce a sequencing library, wherein each of said plurality of second strand primers comprises a 5′ universal primer sequence, a 3′ sequence complementary to a sequence of said first strand cDNA, and a sided sequence (SS), wherein said plurality of second strand primers is configured to hybridize with said plurality of cDNA molecules thereby extended to produce second strand cDNA molecules that comprise unique truncation sites of said plurality of cDNA molecule.

In one aspect, disclosed herein is a system comprising: a plurality of beads; a plurality of cDNA molecules, wherein each of said plurality of beads comprises a first strand of a cDNA molecule of said plurality of cDNA molecules attached thereto; and a plurality of second strand primers for performing second strand synthesis of said plurality of cDNA molecules to produce a sequencing library, wherein each of said plurality of second strand primers comprises a 5′ universal primer sequence, a 3′ complementary to a sequence of said first strand cDNA, and a sided sequence (SS) of 2-5 bases, wherein said plurality of second strand primers is configured to produce a truncation site of a second strand of a cDNA molecule of said plurality of cDNA molecules during said second strand synthesis.

In one aspect, disclosed herein is a system comprising (a) a plurality of cDNA molecules, wherein each of said plurality comprises, in 5′ to 3′ direction, a universal primer sequence, a sided sequence (5′ SS), a target sequence or fragment thereof, a sample barcode, s sided sequence (3′SS), and a universal primer sequence, wherein the cDNA molecules optionally comprise one or more of a random sequence, a specific sequence, and a poly(dA) sequence; and (b) a plurality of indexing primers comprising an adaptor sequence, an index sequence for library indexing, a sided sequences (SS), and a universal primer sequence.

In one aspect, disclosed herein is a system comprising: a plurality of second strand primers for performing second strand synthesis of a plurality of cDNA molecules to produce a sequencing library, wherein each of said plurality of second strand primers comprises a 5′ universal primer sequence, a 3′ random template nucleic acid-binding sequence, and a sided sequence (SS), wherein said plurality of second strand primers is configured to produce a truncation site of a second strand of a cDNA molecule of said plurality of cDNA molecules during said second strand synthesis; and a plurality of indexing primers comprising, in a 5′-3′ direction, an adaptor sequence, an index sequence for indexing nucleic acid molecules of said sequencing library, and sided sequences (SS) that define a 3′ or a 5′ side of said nucleic acid molecules of said sequencing library.

In one aspect, disclosed herein is a method of detecting or monitoring a disease or condition in a subject, comprising counting nucleic acid molecules of a sample according to a method described herein, wherein said sample comprises one or more copies of nucleic acid sequences of said subject, and wherein said number of template nucleic acid molecules is associated with said disease or condition. In some embodiments, the template nucleic acid molecules encode a protein secreted by T cells. In some embodiments, the template nucleic acid molecules comprise sequences of a complementarity determining region (CDR) from T-cell receptor genes or immunoglobulin genes. In some embodiments, the CDR comprises one or more of CDR1, CDR2, and CDR3. In some embodiments, the disease or condition is a proliferative disease, an autoimmune disease, or an infectious disease.

In one aspect, disclosed herein is a method of assaying a sample bioparticle, comprising counting nucleic acid molecules of a sample according to a method described herein, wherein said sample is obtained by making a copy of one or more nucleic acid sequences in said bioparticle and wherein said bioparticle is a T cell or a B cell. In some embodiments, the bioparticle is a chimeric antigen receptor (CAR)-T cell. In some embodiments, the template nucleic acid molecules comprise sequences of a complementarity determining region (CDR) from T-cell receptor genes. In some embodiments, the template nucleic acid molecules are indicative of contamination of said CAR-T cell. In some embodiments, the template nucleic acid molecules are indicative of clonal lineage of said CAR-T cell. In some embodiments, the method comprises releasing RNA molecules from said cell or bioparticle. In some embodiments, the method comprises performing reverse transcription reaction of said RNA molecules thereby forming said plurality of template nucleic acid molecules. In some embodiments, the bioparticle is obtained from a subject.

In one aspect, disclosed herein is a method of detecting or monitoring a disease or condition in a subject, comprising: obtaining a sample fluid from a subject, wherein said sample fluid comprises a plurality of bioparticles; loading said sample fluid onto a microwell array that comprises a plurality microwells, thereby loading a bioparticle into at least one microwell; releasing one or more target nucleic acid molecules from said bioparticle; performing reverse transcription of said target nucleic acid molecules thereby producing template nucleic acid molecules, wherein each template nucleic acid molecule comprising a copy of a sequence of said target nucleic acid molecules; randomly truncating said template nucleic acid molecules at a truncation base position within said template nucleic acid molecules, wherein said truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position; optionally amplifying at least a portion of said plurality of truncated nucleic acid molecules to produce a plurality of amplified nucleic acid molecules, wherein said truncation base positions are preserved in said plurality of amplified nucleic acid molecules; sequencing at least a portion of said amplified nucleic acid molecules or said truncated nucleic acid molecules to determine a number of unique truncation base positions; and identifying a number of template nucleic acid molecules present in said bioparticle using said number of unique truncation base positions. In another aspect, disclosed herein is a method of detecting or monitoring a disease or condition in a subject, comprising: obtaining a sample fluid from a subject, wherein said sample fluid comprises a plurality of bioparticles; loading said sample fluid onto a microwell array that comprises a plurality microwells, thereby loading a bioparticle into at least one microwell; releasing one or more target nucleic acid molecules from said bioparticle; performing reverse transcription of said target nucleic acid molecules thereby producing template nucleic acid molecules, wherein each template nucleic acid molecule comprising a copy of a sequence of said target nucleic acid molecules; randomly truncating said template nucleic acid molecules at a truncation base position within said template nucleic acid molecules, wherein said truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position; sequencing at least a portion of said truncated nucleic acid molecules to determine a number of unique truncation base positions; and identifying a number of template nucleic acid molecules present in said bioparticle using said number of unique truncation base positions. In some embodiments, the sample fluid comprises blood sample of said subject. In some embodiments, the plurality of bioparticles comprise peripheral blood mononuclear cells (PBMCs). In some embodiments, the plurality of bioparticles comprise engineered cells. In some embodiments, the plurality of bioparticles comprise T cells. In some embodiments, the T cells comprise native T cells, engineered T cells, or both. In some embodiments, the T cells comprise one or more native T cells and one or more chimeric antigen receptor (CAR)-T cells. In some embodiments, the method comprises, after loading said sample fluid, storing said microwell array comprising said bioparticle in said at least one microwell for a period of time. In some embodiments, the period of time is between 1 hour and 30 years.

In one aspect, disclosed herein is a method of assaying a plurality of engineered cells, comprising: obtaining a sample fluid comprising a plurality of engineered cells; loading said sample fluid onto a microwell array that comprises a plurality of microwells, thereby loading an engineered cell into one microwell; releasing one or more target nucleic acid molecules from said engineered cell; producing template nucleic acid molecules, each comprising a copy of a sequence of said target nucleic acid molecules; randomly truncating said template nucleic acid molecules at a truncation base position within said template nucleic acid molecules, wherein said truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position; optionally amplifying at least a portion of said plurality of truncated nucleic acid molecules to produce a plurality of amplified nucleic acid molecules, wherein said truncation base positions are preserved in said plurality of amplified nucleic acid molecules; sequencing at least a portion of said amplified nucleic acid molecules or said truncated nucleic acid molecules to determine a number of unique truncation base positions; and identifying a number of template nucleic acid molecules present in said engineered cell using said number of unique truncation base positions. In another aspect, disclosed herein is a method of assaying a plurality of engineered cells, comprising: obtaining a sample fluid comprising a plurality of engineered cells; loading said sample fluid onto a microwell array that comprises a plurality of microwells, thereby loading an engineered cell into one microwell; releasing one or more template nucleic acid molecules from said engineered cell; randomly truncating said template nucleic acid molecules at a truncation base position within said template nucleic acid molecules, wherein said truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position; sequencing at least a portion of said truncated nucleic acid molecules to determine a number of unique truncation base positions; and identifying a number of template nucleic acid molecules present in said engineered cell using said number of unique truncation base positions. In another aspect, disclosed herein is a method of assaying a plurality of engineered cells, comprising: obtaining a sample fluid comprising a plurality of engineered cells; loading said sample fluid onto a microwell array that comprises a plurality of microwells, thereby loading an engineered cell into one microwell; releasing one or more template nucleic acid molecules from said engineered cell; truncating said template nucleic acid molecules at a truncation base position within said template nucleic acid molecules, wherein said truncating comprises performing a selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position; sequencing at least a portion of said truncated nucleic acid molecules to determine a number of unique truncation base positions; and identifying a number of template nucleic acid molecules present in said engineered cell using said number of unique truncation base positions. In some embodiments, the template nucleic acid molecules are randomly truncated. In some embodiments, truncating comprises performing a random selection of said truncation base position among a plurality of base positions of said template nucleic acid molecules and making a copy of at least a portion of said template nucleic acid molecules. In some embodiments, the engineered cells comprise exogenous nucleic acid sequences. In some embodiments, the template and/or target nucleic acid molecules comprise said exogenous nucleic acid sequences. In some embodiments, the engineered cells lack one or more knock-out sequences. In some embodiments, the template and/or target nucleic acid molecules lack said knock-out sequences. In some embodiments, the template and/or target nucleic acid molecules comprise said knock-out sequences. In some embodiments, the method comprises, after loading said sample fluid, storing said microwell array comprising said engineered cell in said at least one microwell for a period of time. In some embodiments, the period of time is between 1 hour and 30 years. In some embodiments, the engineered cells comprise engineered immune cells or engineered stem cells. In some embodiments, the engineered cells comprise engineered protein-secreting cells. In some embodiments, the engineered cells comprise engineered T cells, engineered B cells, or a combination thereof. In some embodiments, the engineered cells comprise chimeric antigen receptor (CAR)-T cells. In some embodiments, the template nucleic acid molecules comprise RNA molecules of said engineered cell. In some embodiments, the template nucleic acid molecules encode a sequence of an immune receptor that is a T-cell receptor (TCR), a B-cell receptor (BCR), a cytokine receptor, a chemokine receptor, a major histocompatibility complex (MHC) class I molecule, a MHC class II molecule, a Toll-like receptor, a killer activation receptor (KAR), a killer-cell immunoglobulin-like receptor (KIR), or an integrin. In some embodiments, the template nucleic acid molecules encode a sequence of a complementarity determining region (CDR) from T-cell receptor genes or immunoglobulin genes. In some embodiments, the CDR comprises one or more of CDR1, CDR2, and CDR3. In some embodiments, the template nucleic acid molecules are indicative of clonal lineage of said engineered cells. In some embodiments, said target nucleic acid molecules are RNA molecules and said template nucleic acid molecules are cDNA molecules.

In one aspect, disclosed herein is a method for counting target mRNA nucleic acid molecules of a single cell sample, comprising: (a) isolating a single cell sample; (b) releasing target mRNA nucleic acid molecules from said single cell sample; (c) capturing said target nucleic acid molecules onto a barcoded bead that is associated with said single cell sample; (d) making first strand cDNA molecules by performing reverse transcription of said target mRNA nucleic acid molecules, wherein said first strand cDNA molecules each comprises a copy of a sequence of said target mRNA molecules; (e) randomly truncating said first strand cDNA molecules at a truncation base position within said plurality of first strand cDNA molecules, wherein said truncating comprises randomly attaching a second strand synthesis primer to the first strand cDNA molecules and extending the synthesis primer, thereby producing a plurality of second strand cDNA molecules each preserving the base position at which the second strand synthesis primer is attached; (f) amplifying at least a portion of said second strand cDNA molecules to produce a plurality of amplified nucleic acid molecules, wherein said truncation base positions are preserved in said amplified nucleic acid molecules; (g) sequencing at least a portion of said plurality of amplified nucleic acid molecules to produce a plurality of sequencing reads, wherein said truncation base positions are preserved in said plurality of sequencing reads; (h) aligning at least a portion of said plurality of sequencing reads to a reference sequence, thereby producing a plurality of aligned sequencing reads; and (i) correlating a number of target mRNA molecules present in said single cell using truncation locations of said plurality of aligned sequencing reads, thereby counting target mRNA nucleic acid molecules. In some embodiments, the first strand cDNA molecules comprise a universal primer sequence, a sided sequence that is configured to establish directionality, a sample barcode, a poly(dT) sequence, and a sequence that comprises a copy of at least a portion of the target mRNA molecule. In some embodiments, the first strand cDNA molecules comprise a universal primer sequence, a sided sequence that is configured to establish directionality, a sample barcode, a sequence that is complementary to a sequence of the target mRNA, and a sequence that comprises a copy of at least a portion of the target mRNA molecule. In some embodiments, the second strand synthesis primer comprise a universal primer sequence, a sided sequence that is configured to establish directionality, and a sequence that is complementary to a sequence of the first strand cDNA molecule. In some embodiments, the sequence that is complementary to a sequence of the first strand cDNA molecule is a random sequence. In some embodiments, each of the sided sequences is independently 5 to 9 bases in length.

In another aspect, the present disclosure provides a system for counting nucleic acid molecules of a sample, comprising: a controller comprising one or more computer processors; and a support operatively coupled to said controller; wherein said one or more computer processors are individually or collectively programmed to: (a) direct the obtaining of a sample comprising a plurality of template nucleic acid molecules; (b) direct the random truncating each of said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules; (c) direct the amplifying of at least a portion of said plurality of truncated nucleic acid molecules to produce a plurality of amplified nucleic acid molecules, wherein said truncation base positions are preserved in said amplified nucleic acid molecules; (d) sequence at least a portion of said plurality of amplified nucleic acid molecules to produce a plurality of sequencing reads, wherein each of said plurality of sequencing reads comprises a truncation location corresponding to said truncation base position of said corresponding amplified nucleic acid molecule; (e) align at least a portion of said plurality of sequencing reads to a reference sequence, thereby producing a plurality of aligned sequencing reads; and (f) identify a number of template nucleic acid molecules present in said sample using truncation locations of said plurality of aligned sequencing reads.

In another aspect, the present disclosure provides a system for counting nucleic acid molecules of a sample, comprising: a controller comprising one or more computer processors; and a support operatively coupled to said controller; wherein said one or more computer processors are individually or collectively programmed to: (a) direct the obtaining of a sample comprising a plurality of template nucleic acid molecules; (b) direct the random truncating of said plurality of template nucleic acid molecules at a truncation base position within said plurality of template nucleic acid molecules, wherein said truncating comprises performing a random selection of said truncation base position among a plurality of base positions of said template nucleic acid molecule and making a copy of at least a portion of said template nucleic acid molecules, thereby producing a plurality of truncated nucleic acid molecules; (c) direct the amplifying of at least a portion of said plurality of truncated nucleic acid molecules to produce a plurality of amplified nucleic acid molecules, wherein said truncation base positions are preserved in said plurality of amplified nucleic acid molecules; (d) sequence at least a portion of said amplified nucleic acid molecules to determine a number of unique truncation base positions present in said at least a portion of said amplified nucleic acid molecules; and (e) identify a number of template nucleic acid molecules present in said sample using said number of unique truncation base positions.

In another aspect, the present disclosure provides a non-transitory computer-readable medium comprising machine-executable code that, upon execution by a computer processor, implements a method for counting nucleic acid molecules of a sample, said method comprising: (a) directing the obtaining of a sample comprising a plurality of template nucleic acid molecules; (b) directing the random truncating each of said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules; (c) directing the amplifying of at least a portion of said plurality of truncated nucleic acid molecules to produce a plurality of amplified nucleic acid molecules, wherein said truncation base positions are preserved in said amplified nucleic acid molecules; (d) sequencing at least a portion of said plurality of amplified nucleic acid molecules to produce a plurality of sequencing reads, wherein each of said plurality of sequencing reads comprises a truncation location corresponding to said truncation base position of said corresponding amplified nucleic acid molecule; (e) aligning at least a portion of said plurality of sequencing reads to a reference sequence, thereby producing a plurality of aligned sequencing reads; and (f) identifying a number of template nucleic acid molecules present in said sample using truncation locations of said plurality of aligned sequencing reads.

In another aspect, the present disclosure provides a non-transitory computer-readable medium comprising machine-executable code that, upon execution by a computer processor, implements a method for counting nucleic acid molecules of a sample, said method comprising: (a) directing the obtaining of a sample comprising a plurality of template nucleic acid molecules; (b) directing the random truncating of said plurality of template nucleic acid molecules at a truncation base position within said plurality of template nucleic acid molecules, wherein said truncating comprises performing a random selection of said truncation base position among a plurality of base positions of said template nucleic acid molecule and making a copy of at least a portion of said template nucleic acid molecules, thereby producing a plurality of truncated nucleic acid molecules; (c) directing the amplifying of at least a portion of said plurality of truncated nucleic acid molecules to produce a plurality of amplified nucleic acid molecules, wherein said truncation base positions are preserved in said plurality of amplified nucleic acid molecules; (d) sequencing at least a portion of said amplified nucleic acid molecules to determine a number of unique truncation base positions present in said at least a portion of said amplified nucleic acid molecules; and (e) identifying a number of template nucleic acid molecules present in said sample using said number of unique truncation base positions.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which exemplary embodiments are utilized, and the accompanying drawings of which:

FIGS. 1A and 1B show examples of workflows for counting nucleic acid molecules of a sample based on truncation locations, in accordance with disclosed embodiments.

FIG. 2 shows an example of a second strand synthesis workflow for converting 3′ barcoded first strand cDNA molecules into a sequencing library, by leveraging second strand synthesis for the addition of a 5′ universal primer sequence (UPS), in accordance with disclosed embodiments.

FIG. 3 shows an example of a second strand synthesis workflow for converting 3′-barcoded first strand cDNA molecules into a sequencing library that maintains unique truncation site in the final sequencing library, in accordance with disclosed embodiments.

FIG. 4 shows an example of a makeup of first and second strand synthesis primers and sequencing primers for a workflow that maintains unique truncation sites, in accordance with disclosed embodiments.

FIG. 5 shows an example of workflow timelines for a conventional workflow and a shortened workflow for sequencing library preparation, in accordance with disclosed embodiments.

FIG. 6 shows a schematic depicting depletion or enrichment of specific transcript sequences in a final sequencing library, by leveraging blocking oligonucleotides during second strand synthesis, in accordance with disclosed embodiments.

FIG. 7 illustrates a computer system that is programmed or otherwise configured to implement methods provided herein.

FIGS. 8A and 8B show an example comparison of gene and transcript counting, respectively, using unique molecular indices or truncation mapping site on same sequencing data, in accordance with disclosed embodiments.

FIGS. 9A and 9B show example plots of gene and transcript yields per cell, respectively, as a function of sequencing read depth from libraries generated with the standard second strand synthesis protocol or the truncated protocol, in accordance with disclosed embodiments.

FIGS. 10A and 10B show the gene and transcript per cell yields respectively from single cell libraries employing unique molecular identifiers or truncation site as the molecule counter.

FIG. 10C displays the transcript count as determined by UMI analysis for each cellular barcode as a function of the transcript count from the same barcodes as determined by truncation mapping. A perfect 1:1 match is plotted as a dashed line.

FIGS. 11A and 11B illustrate an exemplary second strand synthesis primer (FIG. 11A) and an exemplary first strand synthesis primer (FIG. 11B), respectively.

### DETAILED DESCRIPTION

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed. For example, the amount “about 10” includes amounts from 8 to 12.

The term “substantially” as used herein can refer to a value approaching 100% of a given value. In some embodiments, the term can refer to an amount that may be at least about 90%, 91%, 92%, 93%, 94%, 95%, 96%, 97%, 98%, 99%, 99.9%, or 99.99% of a total amount. In some embodiments, the term can refer to an amount that may be about 100% of a total amount.

The term “copy,” in the context of a copy of a nucleic acid, refers to either the complement of the initial nucleic acid, the reverse complement of the initial nucleic acid, or a nucleic acid that has the same nucleotide sequence as the initial nucleic acid.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and is sufficiently long to prime the synthesis of the desired extension product in the presence of the inducing agent. The exact length of the primer will depend upon many factors, including temperature, source of primer and use of the method. For example, for some applications, depending on the complexity of the target sequence, the oligonucleotide primer may contain 5-50, or 15-25, or more nucleotides, although it may contain fewer nucleotides.

As used herein, in the context of nucleic acids, the terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some of the base pairs are complementary. The bases that are not complementary are “mismatched.” Complementarity may also be complete (i.e., 100%), if all the base pairs of the duplex region are complementary. The term complementarity also encompasses reverse complement.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) and/or deoxyribonucleotide monomers. An oligonucleotide may be 2 to 20, 5 to 25, 10 to 20, 21 to 30, 31 to 40, 41 to 50, 51 to 60, 61 to 70, 71 to 80, 80 to 100, 100 to 150 or 150 to 200 nucleotides in length, for example.

The term “mRNA” or sometimes refer by “mRNA molecule” or “mRNA transcript” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing can include splicing, editing and degradation. As used herein, a nucleic acid derived from an mRNA refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA and detection of such derived products is indicative of the presence and/or abundance of the original mRNA in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

### Systems and Methods for Counting Nucleic Acids Molecules

Counting Unique Molecules Based on Truncation Mapping Site

RNA-sequencing (RNA-seq) has become a mainstay technique for measuring the expression of genes in a sample, including down to a single cell. A variety of high-throughput approaches can be used to perform single-cell RNA-seq analysis. Most such approaches may revolve around the addition of a unique barcode or unique molecular identifier (UMI) to the 3′ end of all transcripts derived from a single cell during reverse transcription. These 3′-barcoded libraries are then typically amplified, and fragmented into a proper size suitable for use in a sequencing library. Next, adaptor sequences may be attached to the 3′-barcoded library fragments for sequencing on commercial platforms (e.g., Illumina). The plurality of sequencing reads may then be grouped by each individual sequencing read's barcode or UMI to identify the transcripts captured from each original cell. To ensure reliable and accurate further downstream manipulation and processing of these sequencing libraries, it may be critical to require that the link between the 3′ barcode and the transcript sequence be maintained; otherwise, the cellular origin of a given transcript may be lost.

Conventional RNA-seq methods may rely on quantifying or determining a number of sequencing reads that mapped or aligned to each transcript, and optionally normalized to a length of the transcript, to estimate the relative frequency of each transcript in the original RNA sample. However, such approaches may only provide a relative amount of each initial template RNA molecule, rather than an exact count. Further, such approaches may be susceptible to error due to a number of different biases that may be introduced during operations such as preparation of sequencing libraries, amplification, sequencing, and base calling. Some techniques may be used to accurately count an exact number of molecules in the original RNA sample. This may be typically done by attaching a unique DNA sequence (e.g., a Unique Molecular Index or UMI) to each initial template RNA molecule in a sample prior to amplification. After amplifying the template RNA molecules and sequencing the amplified molecules, the number of unique UMIs associated with sequencing reads that map to each transcript, rather than the sequencing reads themselves, may be quantified or counted, thereby producing an absolute count for the number of each transcript present in the original sample. Such molecule counting may be critical for accurate measurements of expressed transcripts for low input libraries, particularly those derived from single cells. Therefore, 3′-barcoding strategies may typically implement a molecule counting method.

Though methods of molecular counting by UMI generally yield an accurate quantification of the expression profiles of transcripts within a sample, such methods may not be without error. For example, erroneous molecular counts may be obtained in cases where, for example, the UMI sequence is changed in only a portion of the progeny polynucleotides of a given molecule due to base misincorporation during PCR or sequencing error (e.g., errors in base calling). Various filtering methods may be used to identify these mistakes, such as collapsing UMI sequences that have a small Hamming distance in sequence space. However, filtering methods may be imperfect and still produce errors, since they may rely on identifying the origin of molecules by their UMI sequence.

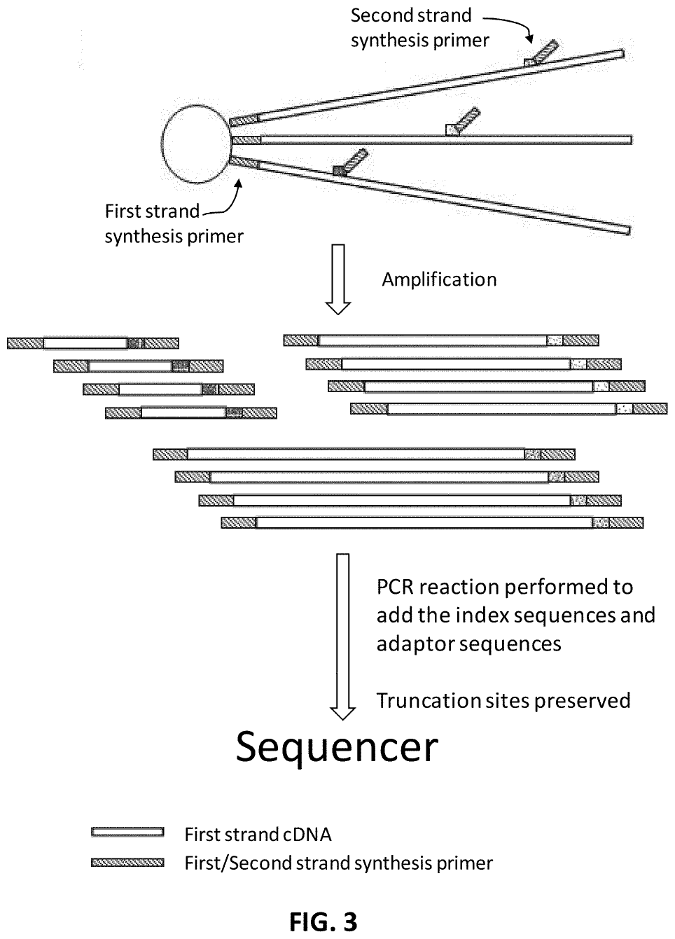

The present disclosure provides methods and systems comprising algorithms for nucleic acid (e.g., RNA or DNA) molecule counting that can be applied in isolation or in combination with UMI to produce a more accurate transcript count with lower error rates. The method can rely on producing a uniquely truncated version of each transcript or the cDNA derived therefrom, during reverse transcription or second strand synthesis. The truncation of each original template nucleic acid molecule (e.g., transcript) can be introduced prior to any amplification of the nucleic acid molecules (e.g., by PCR). This can ensure that progeny polynucleotides of a given molecule contain the same truncation site (e.g., at the same nucleotide position among the polynucleotide). For example, when the truncation site is created during second strand cDNA synthesis, it can refer to the base position where the second strand primer attaches to the first strand cDNA (e.g., as illustrated in FIG. 3).

Further, the present disclosure provides methods of generating sequencing libraries that maintain the unique truncation site in the final sequencing library for each transcript. In some embodiments, after sequencing, the truncation site for each read mapping to a given transcript is identified and quantified. The number of unique mapping sites for each transcript can be used to estimate the number of transcripts present in the original sample of template nucleic acid molecules. In some embodiments, the herein provided sequencing library contains directionality information of the template or target nucleic acid molecules.

In some embodiments, described herein is a method of counting nucleic acid molecules (e.g., mRNAs) of a single cell sample and the method comprises one or more steps selected from (a) RNA capture, (b) first strand cDNA synthesis, (c) 2^{nd} strand cDNA synthesis and truncation mapping sites establishment, (d) amplification of 2^{nd} strand cDNAs, (e) PCR reactions, and (f) sequencing. In an RNA capture step, mRNAs from a single cell can be captured onto a barcoded bead containing a first strand synthesis primer. In first strand cDNA synthesis, the first strand synthesis primer can be extended, thereby generating the first strand cDNA (and making a copy of at least a portion the mRNA). In second strand cDNA synthesis, a 2nd strand synthesis primer (comprising a randomer and a universal primer sequence) can be randomly attached to the first strand cDNA, thereby creating a unique truncation site for each 2^{nd} strand cDNA. During amplification, the second strand cDNA can be amplified while preserving the unique truncation sites in the progenies. The method can comprise one, two, or more PCR reactions. The first PCR reaction can be a suppression PCR. The second PCR reaction can operate to add index sequences and adaptor sequences to the progenies while preserving the unique truncation sites in the progenies. The amplified progenies can be sequenced. The reads can be aligned to a reference sequence. The number of mRNA molecules in the single cell sample can then be correlated with the number of unique truncation sites in the reads.

In some embodiments, a method described herein is illustrated in scheme 1.

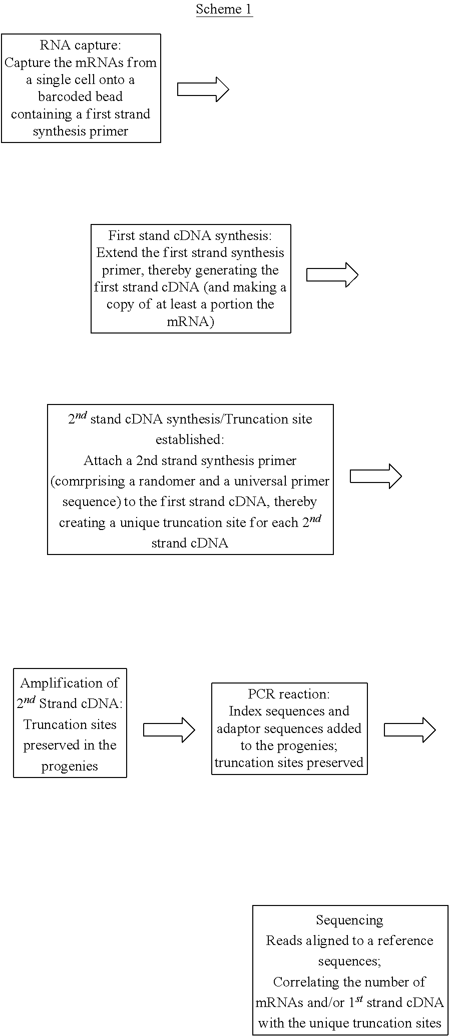

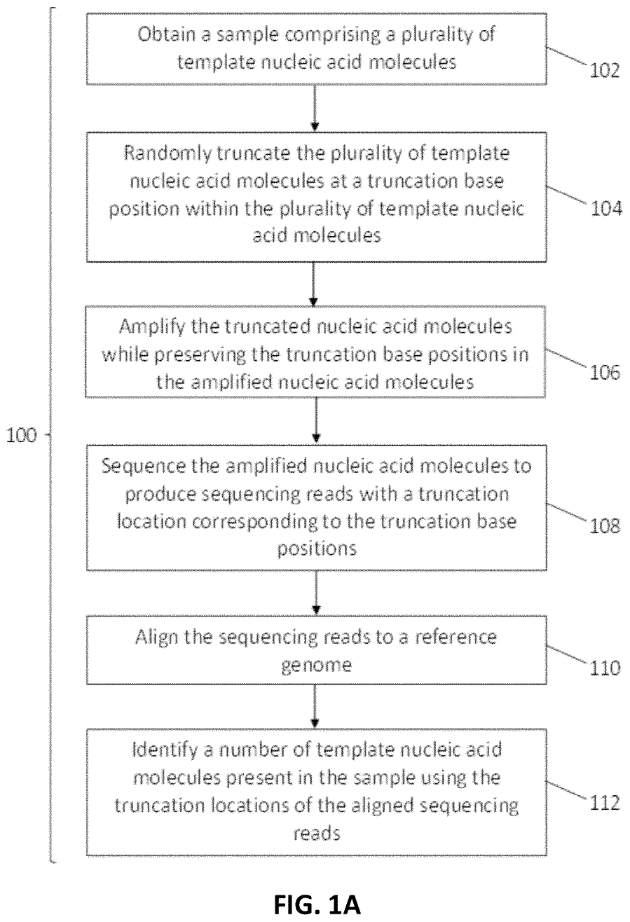

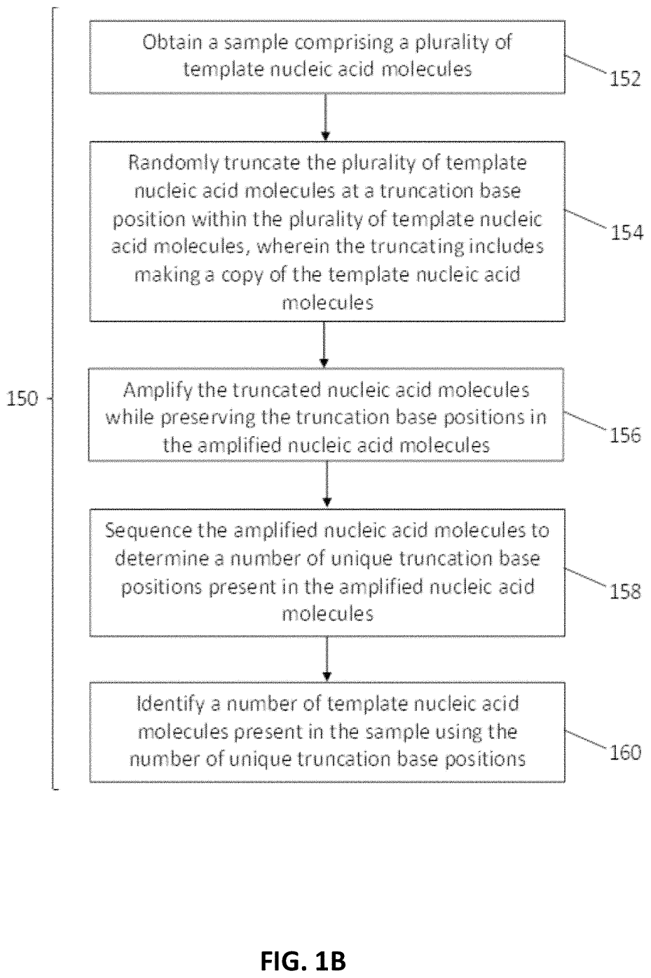

FIGS. 1A and 1B show examples of workflows for counting nucleic acid molecules (e.g., mRNAs) of a sample such as a single cell based on truncation locations, in accordance with disclosed embodiments.

In an aspect, the present disclosure provides a method for counting nucleic acid molecules of a sample. In some embodiments, the method comprises obtaining a sample comprising a plurality of template nucleic acid molecules. In some embodiments, the method comprises randomly truncating said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules. In some embodiments, the truncation base position is preserved in said truncated nucleic acid molecules. In some embodiments, the method comprises amplifying at least a portion of said plurality of truncated nucleic acid molecules to produce a plurality of amplified nucleic acid molecules, wherein said truncation base positions are preserved in said amplified nucleic acid molecules. In some embodiments, the method comprises sequencing at least a portion of said plurality of amplified nucleic acid molecules or truncated nucleic acid molecules to produce a plurality of sequencing reads, wherein each of said plurality of sequencing reads comprises a truncation location corresponding to said truncation base position of said corresponding amplified nucleic acid molecule or truncated nucleic acid molecules. In some embodiments, the method comprises sequencing at least a portion of said plurality of amplified nucleic acid molecules to produce a plurality of sequencing reads, wherein each of said plurality of sequencing reads comprises a truncation location corresponding to said truncation base position of said corresponding amplified nucleic acid molecules. In some embodiments, the method comprises sequencing at least a portion of said plurality of truncated nucleic acid molecules to produce a plurality of sequencing reads, wherein each of said plurality of sequencing reads comprises a truncation location corresponding to said truncation base position of said corresponding truncated nucleic acid molecules. In some embodiments, the method comprises aligning at least a portion of said plurality of sequencing reads to a reference sequence, thereby producing a plurality of aligned sequencing reads. In some embodiments, the method comprises identifying a number of template nucleic acid molecules present in said sample using truncation locations of said plurality of aligned sequencing reads. In some embodiments, the method comprises: (a) obtaining a sample comprising a plurality of template nucleic acid molecules; (b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules; (c) optionally amplifying at least a portion of said plurality of truncated nucleic acid molecules to produce a plurality of amplified nucleic acid molecules, wherein said truncation base positions are preserved in said amplified nucleic acid molecules; (d) sequencing at least a portion of said plurality of amplified nucleic acid molecules or truncated nucleic acid molecules to produce a plurality of sequencing reads, wherein each of said plurality of sequencing reads comprises a truncation location corresponding to said truncation base position of said corresponding amplified nucleic acid molecule or said truncated nucleic acid molecules; (e) aligning at least a portion of said plurality of sequencing reads to a reference sequence, thereby producing a plurality of aligned sequencing reads; and (f) identifying a number of template nucleic acid molecules present in said sample using truncation locations of said plurality of aligned sequencing reads.

FIG. 1A illustrates an example workflow of a method 100 for counting nucleic acid molecules of a sample based on truncation locations, in accordance with disclosed embodiments. The method 100 can comprise obtaining a sample comprising a plurality of template nucleic acid molecules, e.g., cDNAs (as in operation 102). Next, the method 100 can comprise randomly truncating the plurality of nucleic acid molecules at a truncation base position within the plurality of template nucleic acid molecules (as in operation 104). Next, the method 100 can comprise amplifying the truncated nucleic acid molecules while preserving the truncation base positions in the amplified nucleic acid molecules (as in operation 106). Next, the method 100 can comprise sequencing the amplified nucleic acid molecules to produce sequencing reads within a truncation location corresponding to the truncation base positions (as in operation 108). Next, the method 100 can comprise aligning the sequencing reads to a reference genome (as in operation 110). For example, the reference genome can be a human genome or a portion thereof. Next, the method 100 can comprise identifying a number of template nucleic acid molecules present in the sample using the truncation locations of the aligned sequencing reads (as in operation 112).

In another aspect, the present disclosure provides a method for counting nucleic acid molecules of a sample. In some embodiments, the method comprises obtaining a sample comprising a plurality of template nucleic acid molecules. In some embodiments, the template nucleic acid molecules are 1^{st} strand cDNAs. In some embodiments, the sample comprises one or more barcoded beads with the cDNA molecules attached to the beads. In some embodiments, the method comprises randomly truncating said plurality of template nucleic acid molecules at a truncation base position within said plurality of template nucleic acid molecules, wherein said truncating comprises performing a random selection of said truncation base position among a plurality of base positions of said template nucleic acid molecule and making a copy of at least a portion of said template nucleic acid molecules, thereby producing a plurality of truncated nucleic acid molecules. In some embodiments, the truncation base position is preserved in said truncated nucleic acid molecules. In some embodiments, the method comprises amplifying at least a portion of said plurality of truncated nucleic acid molecules to produce a plurality of amplified nucleic acid molecules, wherein said truncation base positions are preserved in said plurality of amplified nucleic acid molecules. In some embodiments, the method comprises sequencing at least a portion of said amplified nucleic acid molecules to determine a number of unique truncation base positions present in said at least a portion of said amplified nucleic acid molecules. In some embodiments, the method comprises identifying a number of template nucleic acid molecules present in said sample using said number of unique truncation base positions. In some embodiments, the method comprises: (a) obtaining a sample comprising a plurality of template nucleic acid molecules; (b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule and making a copy of at least a portion of said template nucleic acid molecules, thereby producing a plurality of truncated nucleic acid molecules; (c) optionally amplifying at least a portion of said plurality of truncated nucleic acid molecules to produce a plurality of amplified nucleic acid molecules, wherein said truncation base positions are preserved in said plurality of amplified nucleic acid molecules; (d) sequencing at least a portion of said amplified nucleic acid molecules or truncated nucleic acid molecules to determine a number of unique truncation base positions; and (e) identifying a number of template nucleic acid molecules present in said sample using said number of unique truncation base positions.

FIG. 1B illustrates an example workflow of a method 150 for counting nucleic acid molecules of a sample based on truncation locations, in accordance with disclosed embodiments. The method 150 can comprise obtaining a sample comprising a plurality of template nucleic acid molecules (as in operation 152). Next, the method 150 can comprise randomly truncating the plurality of nucleic acid molecules at a truncation base position within the plurality of template nucleic acid molecules (as in operation 154). For example, the truncating can include making a copy of the template nucleic acid molecules. Next, the method 150 can comprise amplifying the truncated nucleic acid molecules while preserving the truncation base positions in the amplified nucleic acid molecules (as in operation 156). Next, the method 150 can comprise sequencing the amplified nucleic acid molecules to determine a number of unique truncation base positions present in the amplified nucleic acid molecules (as in operation 158). Next, the method 150 can comprise identifying a number of template nucleic acid molecules present in the sample using the number of unique truncation base positions (as in operation 160).

The truncating can be performed by cleaving the plurality of template nucleic acid molecules. For example, the cleaving can be performed by base-catalyzed hydrolysis, ultrasonic shearing, or partial enzymatic degradation, of the plurality of template nucleic acid molecules. In some embodiments, the truncating comprises making a copy of at least a portion of the plurality of template nucleic acid molecules. In some embodiments, the copy comprises a sequence identical to a sequence of the template nucleic acid molecules. In some embodiments, the copy comprises a sequence complementary to a sequence of the template nucleic acid molecules.

In some embodiments, at least a portion of the plurality of sequencing reads can be aligned to a reference sequence, thereby producing a plurality of aligned sequencing reads. For example, the reference genome can be a human genome or a portion thereof. In some embodiments, at least a portion of the amplified nucleic acid molecules can be processed to produce a sequencing library. The sequencing library can be produced such as to preserve the truncation base positions of the molecules of the sequencing library.

In some embodiments, the plurality of template nucleic acid molecules comprises deoxyribonucleic acid (DNA) molecules. In some embodiments, said plurality of template nucleic acid molecules comprises ribonucleic acid (RNA) molecules. In some embodiments, the plurality of template nucleic acid molecules comprises complementary DNA (cDNA) molecules. For example, the cDNA molecules can be derived from RNA molecules (e.g., by reverse transcription). In some embodiments, reverse transcription of a plurality of target nucleic acid molecules in the sample is performed to generate a plurality of template nucleic acid molecules.

In some embodiments, a sample described herein comprises copies of nucleic acids that are obtained from a bioparticle such as single cell. For example, a cellular sample containing a plurality of cells can be isolated, partitioned, or fractionated across a plurality of phase partitions, so as to obtain sub-samples containing single cells. The partitioning or fractionation can be performed using microwells (e.g., a microwell array) or droplets, which are sized to perform single-cell or substantially single-cell isolation. The single-cell samples can then be processed to extract the plurality of target nucleic acid molecules contained therein (such as mRNA molecules). In some embodiments, the plurality of target nucleic acid molecules can be processed and released from the sample. In some embodiments, the target nucleic acid molecules are RNA molecules and the template nucleic acid are cDNA molecules. In some embodiments, the plurality of template nucleic acid molecules (e.g., first strand cDNA molecules) is pooled across the plurality of phase partitions, for further downstream processing. In some embodiments, the template nucleic acid molecules are first strand cDNA molecules formed via reverse transcription from target RNA molecules. In some embodiments, the template nucleic acid molecules are derived from target nucleic acid molecules of a single cell.

In some embodiments, the target molecules described herein are RNA molecules from a cellular sample. In some embodiments, the RNA is a messenger RNA (mRNA) or a fragment thereof. The mRNA can be polyadenylated or non-polyadenylated. In some embodiments, the RNA molecules are a population of different mRNAs. In some embodiments, the RNA is a non-coding RNA (ncRNA). For example, the ncRNA can be long noncoding RNA (IncRNA), long intergenic non-coding RNA (lincRNA), micro RNA (miRNA), small interfering RNA (siRNA), Piwi-interacting RNA (piRNA), trans-acting RNA (rasiRNA), ribosomal RNA (rRNA), transfer RNA (tRNA), mitochondrial tRNA (MT-tRNA), small nuclear RNA (snRNA), small nucleolar RNA (snoRNA), SmY RNA, Y RNA, spliced leader RNA (SL RNA), telomerase RNA component (TERC), fragments thereof, or combinations thereof. In some embodiments, the RNA is a transcriptome of a cell or population of cells. The RNA can be derived from eukaryotic, archaeal, or bacterial cells.

The amount of input RNA can vary in a described method. In some embodiments, the processes disclosed herein can amplify low or single cell input quantities of RNA molecules. In some embodiments, the amount of input RNA can be at least about 1 pg, at least about 5 picograms (pg), at least about 10 pg, at least about 20 pg, at least about 50 pg, at least about 100 pg, at least about 200 pg, at least about 500 pg, or more than about 500 pg of RNA. In some embodiments, the amount of input RNA can range from about 10 pg to about 100 pg. In some embodiments, the amount of input RNA is all or a portion of the RNA molecules from a single cell. The quality or integrity of RNA molecules can vary. In some embodiments, the quality of input RNA ranges from low quality (i.e., degraded or fragmented) to high quality (i.e., intact). For example, the quality of total RNA can be estimated on the basis of the ratio of 28S rRNA to 18S rRNA. In some embodiments, the RNA can have a 28S:18S ratio of at least about 2:1, a 28S:18S ratio of at least about 1:1, a 28S:18S ratio of less than about 1:1, or an undetectable 28S:18S ratio.

In some embodiments, a plurality of second strand cDNA molecules is formed from the plurality of template nucleic acid molecules, such that the plurality of second strand cDNA molecules comprises the truncation base positions. For example, the plurality of template nucleic acid molecules can be contacted with a plurality of second strand primers. The plurality of second strand primers can each comprise a 5′ universal primer sequence (UPS) and a 3′ sequence complementary to a sequence of said template nucleic acid. In some embodiments, the 3′ sequence is a random sequence. In some embodiments, the 3′ random sequence hybridizes and binds with a sequence in the template nucleic acid in a site-nonspecific fashion. In some embodiments, the plurality of second strand primers can each comprise a 5′ universal primer sequence (UPS) and a 3′ sequence complementary to the template nucleic acid. In some embodiments, the 3′ sequence of the second strand primer comprises a random sequence. In some embodiments, the 3′ sequence of the second strand primer comprises a random template nucleic acid-binding sequence. In some embodiments, the plurality of second strand primers can be extended to produce the plurality of second strand cDNA molecules. For example, random transposon insertion of the plurality of second strand cDNA molecules can be performed to randomly fragment the plurality of second strand cDNA molecules. For another example, a complex of the first strand cDNA and the template RNA can be fragmented. In some embodiments, the second strand cDNA molecules are fragmented by random transposon insertion. In some embodiments, the cDNA-RNA hybrid are fragmented by random transposon insertion.

In some embodiments, the 3′ sequence of the second strand primer comprises 1, 2, 3, 4, 5, 6, 7, 8, 9, 10, 11, 12, 13, 14, 15, 16, or more than 16 bases. For example, the 3′ random template nucleic acid-binding sequence can comprise 9 or 10 bases. In some embodiments, the 3′ random template nucleic acid-binding sequence can comprise 5-12 bases. The 3′ random template nucleic acid-binding sequence can be linked on its 5′ side to a universal primer sequence. In some embodiments, the second strand primers each comprise a 5′ sided sequence (SS). For example, the 5′ SS can comprise 1, 2, 3, 4, 5, 6, 7, 8, 9, or 10 bases. In some embodiments, the 5′ SS of each of the second strand primers are the same. In some embodiment, the 5′ SS comprises 2 to 5 bases. In some embodiment, the 5′ SS comprises 5-9 bases. In some embodiment, the 5′ SS comprises 7 to 15 bases or 10 to 25 bases. In some embodiments, the 5′ SS flanks the universal primer sequence. The template nucleic acid molecules can comprise a 3′ sided sequence. In some embodiments, the 3′ SS of each of the template nucleic acid are the same. For example, the 3′ SS can comprise 1, 2, 3, 4, 5, 6, 7, 8, 9, or 10 bases. In some embodiment, the 3′ SS comprises 2 to 5 bases. In some embodiment, the 3′ SS comprises 5-9 bases. In some embodiment, the 3′ SS comprises 7 to 15 bases or 10 to 25 bases. In some embodiments, the 3′ SS flanks the universal primer sequence.

In some embodiments, the plurality of target nucleic acid molecules are each tagged with a unique sample barcode among a plurality of sample barcodes. For example, each of the plurality of sample barcodes can comprise a set of one or more nucleotide bases (e.g., 1, 2, 3, 4, 5, 6, 7, 8, 9, 10, 11, 12, 13, 14, 15, 16, or more than 16 nucleotide bases). In some embodiments, the plurality of target nucleic acid molecules is tagged with a sample barcode that is indicative of a sample with which the target nucleic acid molecules are associated. For example, each sample obtained from a different subject can be tagged with a different sample barcode. The sample barcode can be identical among all of the plurality of target nucleic acid molecules in a sample.

In some embodiments, a plurality of chain-terminating nucleotides can be used to perform the random truncation at said truncation base position. For example, the chain-terminating nucleotides can be dideoxynucleotides. The chain-terminating nucleotides can be configured to produce a desired distribution of truncation size among the plurality of truncated nucleic acid molecules. In some embodiments, a 3′ carbon position of the plurality of chain-terminating nucleotides can be chemically labeled to enable chemical ligation of a 5′ universal primer site (UPS) of the template nucleic acid molecules.

In some embodiments, the nucleic acid molecules are amplified using polymerase chain reaction (PCR) amplification. For example, the PCR amplification can comprise suppression PCR amplification. In some embodiments, the nucleic acid molecules are amplified using two or more PCR amplification steps. In some embodiments, the method comprises a suppression PCR and a second PCR amplification that re-establishes the directionality of the sequencing library. In some embodiments, the directionality of the sequencing library is re-established by the presence of the 5′SSs and 3′SSs. The sequencing library can comprise sided sequences (SS) on a 3′ and a 5′ side of nucleic acid molecules of the sequencing library. The SS can be known sequences. The SS can be unique sequences. In some embodiments, all 5′SS is the same in the sequencing library. In some embodiments, all 3′SS is the same in the sequencing library. For example, the sided sequences can have a length of 1, 2, 3, 4, 5, 6, 7, 8, 9, 10, 11, 12, 13, 14, 15, 16, or more than 16 bases. In some embodiments, the SSs can have a length of 2 to 5 bases. In some embodiments, the SSs can have a length of 5 to 9 bases. In some embodiments, the SSs can have a length of 5 to 25 bases. In some embodiments, each of the nucleic acid molecules in the sequencing library has the same 5′ SSs. In some embodiments, each of the nucleic acid molecules in the sequencing library has the same 3′ SSs. In some embodiments, the 5′SS is not identical to the 3′ SS.

In some embodiments, the second PCR amplification comprises amplifying suppression PCR products with indexing primers. The indexing primers can contain, in a 5′-3′ direction, an adaptor sequence, an index sequence for indexing of said sequencing library, and a custom sequencing primer sequence. For example, the custom sequencing primer sequence can comprise a portion of a UPS sequence and a sided sequence that defines a 3′ or a 5′ side of said sequencing library. The custom sequencing primer sequence can have a length of from about 10 to 100 bases, and/or ranges therebetween. In some embodiments, the custom sequencing primer sequence has a length of from about 10 to 100 bases, from about 15 to about 75 bases, from about 20 to about 50 bases, or from about 25 to about 40 bases. In some embodiments, a custom sequencing primer sequence has a length that is 15, 16, 17, 18, 19, 20, 21, 22, 23, 24, 25, 26, 27, 28 29, 30, 31, 32, 33, 34, 35, 36, 37, 38, 39, 40, 41, 42, 43, 44, 45, 46, 47, 48, 49, or 50 bases. In some embodiments, the second PCR amplification comprises using a PCR annealing time of about 1 minute, about 2 minutes, about 3 minutes, about 4 minutes, about 5 minutes, about 6 minutes, about 7 minutes, about 8 minutes, about 9 minutes, about 10 minutes, or more than about 10 minutes. In some embodiments, the second PCR amplification is performed without purification of suppression PCR products of the suppression PCR amplification.

In some embodiments, a number of the plurality of template nucleic acid molecules can be correlated. The correlation can be performed based at least in part on determining a quantitative measure of the plurality of aligned sequencing reads having a same mapping base location.

In some embodiments, the plurality of truncated nucleic acid molecules can be tagged with a non-unique barcode among a plurality of non-unique barcodes. For example, each of the plurality of non-unique barcodes can comprise a set of one or more nucleotide bases. In some embodiments, the plurality of non-unique barcodes comprise barcode sequences of from about 2 to about 100, from about 2 to about 75, from about 2 to about 50, from about 2 to about 25, from about 2 to about 15, or from about 2 to about 10 base. In some embodiments, the plurality of non-unique barcodes comprise barcode sequences of 2, 3, 4, 5, 6, 7, 8, 9, 10, 11, 12, 13, 14, 15, 16, 17, 18, 19, or 20 bases. In some embodiments, each of the non-unique barcodes comprises from about 2 to about 10 bases. The set of non-unique barcodes can comprise, for example, about 10 to about 100 distinct non-unique barcodes. In some embodiments, from about 1% to about 30%, from about 5% to about 20%, or from about 8% to 15% of the plurality of template nucleic molecules are tagged with the non-unique barcode. The non-unique barcodes can comprise, for example, about 10 to about 100 nucleotide bases. The correlation can be performed based at least in part on determining a quantitative measure of the plurality of aligned sequencing reads having a same mapping base location and a same non-unique barcode.

In some embodiments, each of the plurality of template nucleic acid molecules comprises a unique sample barcode among a plurality of sample barcodes. For example, each of the plurality of sample barcodes can comprise a set of one or more nucleotide bases. The set of sample barcodes can comprise, for example, about 5 to about 100 distinct sample barcodes, and/or ranges therebetween. The sample barcodes can comprise, for example, from about 5 to about 200, from about 5 to about 100, from about 10 to about 100, from about 10 to about 50, from about 10 to about 25 nucleotide bases, and/or ranges therebetween. The number of template nucleic acid molecules present in said sample can be identified using a number of the plurality of aligned sequencing reads having a same mapping base location, a same non-unique barcode, and/or a same sample index. For example, the plurality of template nucleic acid molecules (e.g., obtained from the same sample of a subject) can comprise a common sample barcode.

In some embodiments, the method further comprises enriching or depleting the plurality of amplified nucleic acid molecules for one or more target sequences. For example, the plurality of amplified nucleic acid molecules can be depleted for one or more target sequences, such as ribosomal RNA (rRNA) sequences. One or more blocking oligonucleotides can be used, that comprise a target sequence of the target sequences. For example, the plurality of amplified nucleic acid molecules can be enriched for one or more target sequences, such as a variable region in a T-cell or B-cell receptor, a single nucleotide polymorphism (SNP), a splicing junction, or a combination thereof.

In some embodiments, the sequencing comprises whole genome sequencing (WGS), massively parallel sequencing, next-generation sequencing (NGS), paired-end sequencing, etc. The sequencing can be performed at a depth of no more than about 50×, no more than about 45×, no more than about 40×, no more than about 35×, no more than about 30×, no more than about 25×, no more than about 20×, no more than about 18×, no more than about 16×, no more than about 14×, no more than about 12×, no more than about 10×, no more than about 8×, no more than about 6×, no more than about 4×, no more than about 2×, or no more than about 1×.

In some embodiments, the sequencing comprises obtaining a first sequencing read and a second sequencing read. For example, the sample barcode can be captured in the first sequencing read. For example, the truncation location corresponding to the truncation base position can be captured in the second sequencing read. The template nucleic acid molecules can be aligned to the reference sequence according to the first or second sequencing read. In some embodiments, the template nucleic acid molecules can be aligned to the reference sequence according to the second sequencing read. In some embodiments, the non-unique barcodes are captured in the second sequencing read. The second sequencing read can comprise, for example, sequencing from about 10 to 200 bases, from about 10 to about 50 bases, or from about 15 to 35 bases in the template nucleic acid molecules. In some embodiments, the first sequencing read is obtained by sequencing a 3′ side sequence of the template nucleic acid, and the second sequencing read is obtained by sequencing a 5′ side sequence of said template nucleic acid. In some embodiments, the sample is a biological sample (e.g., obtained from a subject).

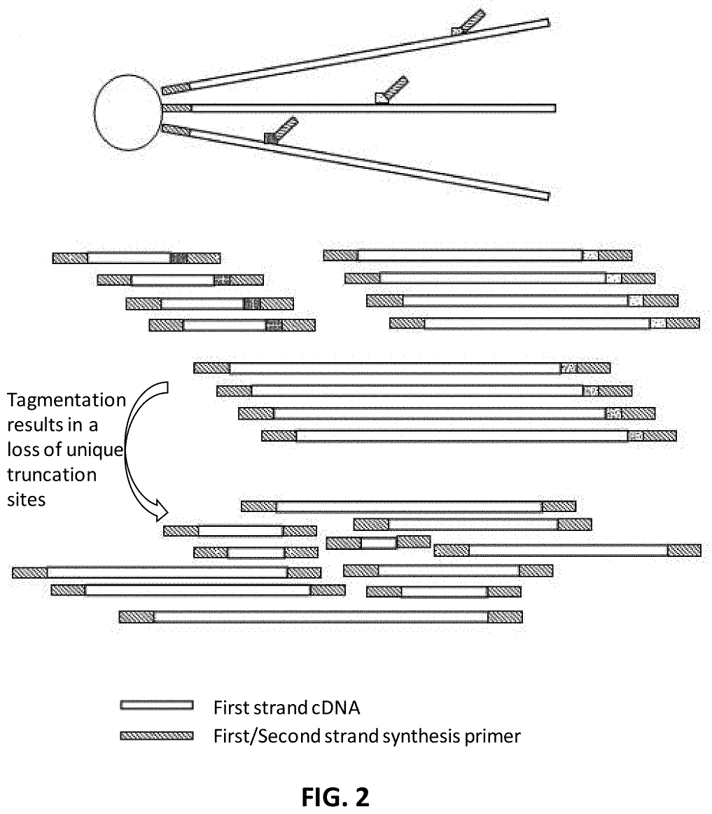

FIG. 2 shows an example of a second strand synthesis workflow for converting 3′ barcoded first strand cDNA molecules into a sequencing library, by leveraging second strand synthesis for the addition of a 5′ universal primer sequence (UPS), in accordance with disclosed embodiments. First, messenger RNA (mRNA) molecules are captured on barcoded poly(dT) beads. Next, the mRNA molecules are converted into first strand cDNA molecules by reverse transcription, thereby forming cDNA-RNA hybrid molecules. Next, the cDNA-RNA hybrid molecules are denatured by adding sodium hydroxide (NaOH), which separates the RNA strand from the cDNA strand, leaving only the cDNA strand attached to the barcoded poly(dT) beads. Next, a random primer with a tail containing a 5′ universal primer sequence (UPS) is used to prime each of the second strand cDNA molecules at random locations. For example, FIG. 2 shows each of three second strand cDNA molecules being primed with the random primer at a different universal primer site, thereby producing a unique truncation site for each molecule. Next, the truncated second strand cDNA molecules are amplified (e.g., by primer extension and PCR). For example, FIG. 2 shows each of the truncated second strand cDNA molecules being amplified into families of progeny polynucleotides, such that each of the progeny polynucleotides maintains its unique mapping site and has the same length within the same family but a different length across different families. The amplified products are purified and then tagmented to yield the final sequencing library. Since the progeny polynucleotides are each tagmented at different sites, the tagmentation step can result in eliminating the original truncation site established by the second strand synthesis reaction, so it may not be possible to determine the molecular lineage of any molecule in the tagmentation library. Therefore, the second strand synthesis workflow shown in FIG. 2 may not preserve or maintain the truncation mapping site of the truncated second strand cDNA molecules.

FIG. 3 shows an example of a second strand synthesis workflow for converting 3′-barcoded first strand cDNA molecules into a sequencing library that maintains unique truncation site in the final sequencing library, in accordance with disclosed embodiments. The sequencing library generation can begin with similar steps as that described in FIG. 2. First, messenger RNA (mRNA) molecules are captured on barcoded poly(dT) beads. Next, the mRNA molecules are converted into first strand cDNA molecules by reverse transcription, thereby forming cDNA-RNA hybrid molecules. Next, the cDNA-RNA hybrid molecules are denatured by adding sodium hydroxide (NaOH), which separates the RNA strand from the cDNA strand, leaving only the cDNA strand attached to the barcoded poly(dT) beads. Next, a random primer with a tail containing a 5′ universal primer sequence (UPS) is used to prime each of the second strand cDNA molecules at random locations. For example, FIG. 3 shows each of three second strand cDNA molecules being primed with the random primer at a different universal primer site, thereby producing a unique truncation site for each molecule. Next, the truncated second strand cDNA molecules are amplified (e.g., by primer extension and PCR). For example, FIG. 3 shows each of the truncated second strand cDNA molecules being amplified into families of progeny polynucleotides, such that each of the progeny polynucleotides maintains its unique mapping site and has the same length within the same family but a different length across different families. Next, instead of tagmenting the sequencing library, a second PCR reaction is performed to add the index sequences and adaptor sequences for the sequencing reaction to the progeny polynucleotide molecules. Through this approach, the unique truncation sites established during the second strand synthesis are maintained in all progeny polynucleotide molecules. This feature can be critical to enabling the use of the site to count the number of original molecules, as described elsewhere herein.

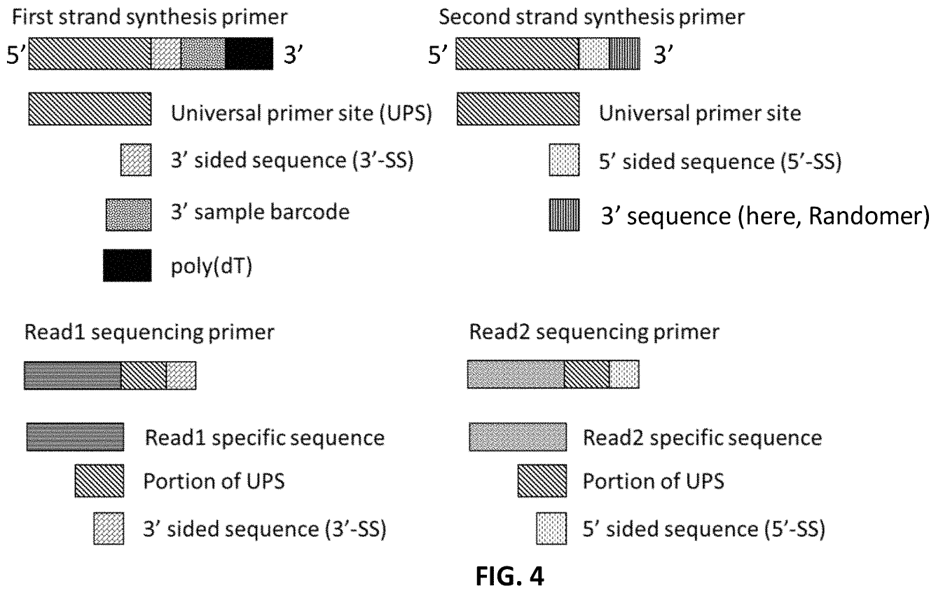

FIG. 4 shows an example of a makeup of first and second strand synthesis primers and sequencing primers for a workflow that maintains unique truncation sites, in accordance with disclosed embodiments.

In some embodiments, a first strand synthesis primer described herein comprises a universal primer site (UPS), a sided sequence (i.e., 3′-SS), a sample barcode (e.g., 3′ sample barcode), and/or a sequence that hybridizes with a target nucleic acid molecule such as an RNA. In some embodiments, a first strand synthesis primer described herein comprises a universal primer site (UPS), a sided sequence (i.e., 3′-SS), a sample barcode (e.g., 3′ sample barcode), and a poly(dT). In some embodiments, a first strand synthesis primer described herein comprises a universal primer site, a sided sequence (i.e., 3′-SS), a sample barcode, and a targeting sequence that hybridizes with a sequence of interest in an RNA. For example, the UPS can contain a length of about 1, 2, 3, 4, 5, 6, 7, 8, 9, 10, 11, 12, 13, 14, 15, 16, or more than 16 bases. In some embodiments, the UPS contains a length of about 15 to 50, about 12 to 20, about 20 to 40, about 20 to 30, or about 20 to 25 bases. In some embodiments, the UPS contains a length of about 20 to 25 bases. In some embodiments, the UPS contains a length of about 20, 21, 22, 23, 24, 25, 26, 27, 28, 29, or 30 bases. For example, the SS on the first strand synthesis primer (i.e., 3′-SS) can contain a length of about 1, 2, 3, 4, 5, 6, 7, 8, 9, 10, 11, 12, 13, 14, 15, 16, or more than 16 bases. In some embodiments, the SS on the first strand synthesis primer has a length of about 2-5 bases. In some embodiments, the SS has a length of about 5-9 bases. In some embodiments, the SS has a length of about 2 to 5, about 5 to 9, about 5 to 12, or about 10 to 20 bases. In some embodiments, the SS has a length of about 5 bases. In some embodiments, the SS has a length of about 6 bases. In some embodiments, the SS has a length of about 7 bases. In some embodiments, the SS has a length of about 8 bases. In some embodiments, the SS has a length of about 9 bases. The sample barcode can contain a suitable number of bases, for example 5 to 50 bases. In some embodiments, the sample barcode has a length of about 5 to 25 bases or any numbers or ranges therebetween. In some embodiments, the sample barcode has a length of about 5 to 15, about 5 to 10, about 6 to 12, about 10 to 20, about 15 to 25, 8 to 15, about 7 to 10, or about 8 to 9 bases. In some embodiments, the sample barcode has a length of about 7 to 10 bases. In some embodiments, the sample barcode has a length of about 8 bases. In some embodiments, the sample barcode has a length of about 9 bases. In some embodiments, the sample barcode has a length of about 10 bases. In some embodiments, the sample barcode has a length of about 5, 6, 7, 8, 9, 10, 11, 12, 13, 14, 15, 16, 17, 18, 19, 20, or more than 20 bases. In some embodiments, the sequence that hybridizes with a target nucleic acid molecule (such as a poly(dT) sequence) has a length of about 7 to 12, 5 to 15, 9 to 10, 4 to 10, 10 to 40, 20 to 40, 25 to 35, or 10 to 50 bases. In some embodiments, the sequence that hybridizes with a target nucleic acid molecule (such as a poly(dT) sequence) has a length of about 25, 26, 27, 28, 29, 30, 31, 32, 33, 34, or 35 bases. In some embodiments, the sequence that hybridizes with a target nucleic acid molecule (such as a poly(dT) sequence) has a length of about 30 bases. In some embodiments, the sequence that hybridizes with a target nucleic acid molecule (such as a poly(dT) sequence) has a length of about 25 bases. In some embodiments, the sequence that hybridizes with a target nucleic acid molecule (such as a poly(dT) sequence) has a length of about 40 bases. In some embodiments, the sequence that hybridizes with a target nucleic acid molecule (such as a poly(dT) sequence) has a length of about 25 to 35 bases.

In some embodiments, a second strand synthesis primer described herein comprises a university primer sequence, a sided sequence (i.e., 5′ SS), and/or a sequence that hybridizes with a first strand cDNA. The sequence that hybridizes with the first strand cDNA can be a random sequence, a semi-random sequence, or a sequence that hybridizes with a sequence of interest in the first strand cDNA. In some embodiments, a second strand synthesis primer comprises a universal primer site, a sided sequence (i.e., 5′-SS), and a random sequence, i.e., a randomer. For example, the UPS can contain a length of about 1, 2, 3, 4, 5, 6, 7, 8, 9, 10, 11, 12, 13, 14, 15, 16, or more than 16 bases. In some embodiments, the UPS contains a length of about 15 to 50, about 12 to 20, about 20 to 40, about 20 to 30, or about 20 to 25 bases. In some embodiments, the UPS contains a length of about 20 to 25 bases. In some embodiments, the UPS contains a length of about 20, 21, 22, 23, 24, 25, 26, 27, 28, 29, or 30 bases. For example, the SS on the second strand synthesis primer (5′-SS) can contain a length of about 1, 2, 3, 4, 5, 6, 7, 8, 9, 10, 11, 12, 13, 14, 15, 16, or more than 16 bases. In some embodiments, the SS on the second strand synthesis primer has a length of about 2-5 bases. In some embodiments, the SS has a length of about 5-9 bases. In some embodiments, the SS has a length of about 2 to 5, about 5 to 9, about 5 to 12, or about 10 to 20 bases. In some embodiments, the SS has a length of about 5 bases. In some embodiments, the SS has a length of about 6 bases. In some embodiments, the SS has a length of about 7 bases. In some embodiments, the SS has a length of about 8 bases. In some embodiments, the SS has a length of about 9 bases. For example, the randomer can contain a length of about 1, 2, 3, 4, 5, 6, 7, 8, 9, 10, 11, 12, 13, 14, 15, 16, or more than 16 bases. In some embodiments, the sequence that hybridizes with the first strand cDNA has a length of about 7 to 12, about 5 to 15, about 7 to 10, about 9 to 10, about 4 to 10, or about 10 to 20 bases. In some embodiments, the sequence that hybridizes with the first strand cDNA has a length of about 5, 6, 7, 8, 9, 10, 11, 12, 13, 14, or 15 bases. In some embodiments, the sequence that hybridizes with the first strand cDNA has a length of about 8 bases. In some embodiments, the sequence that hybridizes with the first strand cDNA has a length of about 9 bases. In some embodiments, the sequence that hybridizes with the first strand cDNA has a length of about 10. In some embodiments, the sequence that hybridizes with the first strand cDNA has a length of about 5 to 15 bases. In some embodiments, the sequence that hybridizes with the first strand cDNA comprises a random sequence and a semi-random sequence. In some embodiments, a randomer comprises a random nucleic sequence. In some embodiments, a randomer hybridizes with a nucleic acid of interest in a site-nonspecific fashion.

The first read (Read1) sequencing primer comprises a Read1 specific sequence, a portion of UPS, and a 3′ sided sequence (3′-SS). For example, the UPS can contain a length of about 1, 2, 3, 4, 5, 6, 7, 8, 9, 10, 11, 12, 13, 14, 15, 16, or more than 16 bases. For example, the 3′-SS can contain a length of about 1, 2, 3, 4, 5, 6, 7, 8, 9, 10, 11, 12, 13, 14, 15, 16, or more than 16 bases. In some embodiments, the 3′-SS has a length of about 16-32 bases. In some embodiments, the 3′-SS has a length of about 7 bases. In some embodiments, the 3′-SS has a length of 5, 6, 7, 8, or 9 bases. In some embodiments, the 3′-SS has a length of 5-9 bases.

The second read (Read2) sequencing primer comprises a Read2 specific sequence, a portion of UPS, and a 5′ sided sequence (5′-SS). For example, the UPS can contain a length of about 1, 2, 3, 4, 5, 6, 7, 8, 9, 10, 11, 12, 13, 14, 15, 16, or more than 16 bases. For example, the 5′-SS can contain a length of about 1, 2, 3, 4, 5, 6, 7, 8, 9, 10, 11, 12, 13, 14, 15, 16, or more than 16 bases. In some embodiments, the 5′-SS has a length of about 16-32 bases. In some embodiments, the 5′-SS has a length of about 7 bases. In some embodiments, the 5′-SS has a length of 5, 6, 7, 8, or 9 bases. In some embodiments, the 5′-SS has a length of 5-9 bases.

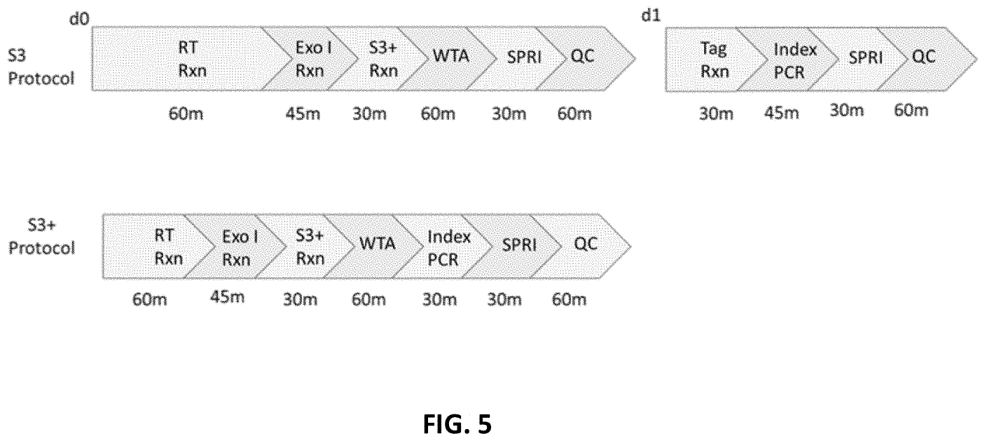

FIG. 5 shows an example of workflow timelines for a conventional workflow and a shortened workflow for sequencing library preparation, in accordance with disclosed embodiments. By leveraging the second strand synthesis reaction to perform the required truncation event instead of a later separate tagmentation reaction, multiple labor intensive and costly steps can be avoided, including tagmentation, a SPRI cleanup, and library quantitation. By eliminating these steps, the entire protocol can easily be completed about 30% faster.

In some embodiments, the conventional workflow (S3 protocol) comprises a reverse transcription (RT) reaction (e.g., about 60 minutes), an Exonuclease I (Exo I) reaction (e.g., about 45 minutes), an S3+ reaction (e.g., about 30 minutes), a whole transcriptome amplification (WTA) step (e.g., about 60 minutes), a solid phase reversible immobilization (SPRI) step (e.g., about 30 minutes), and a quality control (QC) step (e.g., about 60 minutes). In some embodiments, the conventional workflow (S3 protocol) further comprises a tagmentation (tag) reaction (e.g., about 30 minutes), an indexing PCR reaction (e.g., about 45 minutes), an SPRI step (e.g., about 30 minutes), and a quality control (QC) step (e.g., about 60 minutes). Therefore, the conventional workflow (S3 protocol) can take at least about 7.5 hours to complete.

In some embodiments, a method described herein has a shortened workflow compared to a conventional method. In some embodiments, the shortened workflow (S3+ protocol) comprises a reverse transcription (RT) reaction (e.g., about 60 minutes), an Exonuclease I (Exo I) reaction (e.g., about 45 minutes), an S3+ reaction (e.g., about 30 minutes), a whole transcriptome amplification (WTA) step (e.g., about 60 minutes), an indexing PCR reaction (e.g., about 45 minutes), a solid phase reversible immobilization (SPRI) cleanup step (e.g., about 30 minutes), and a library quantitation quality control (QC) step (e.g., about 60 minutes). In some embodiments, the shortened workflow (S3+ protocol) does not include a tagmentation (tag) reaction (e.g., about 30 minutes). In some embodiments, the shortened workflow does not include a subsequent SPRI cleanup (e.g., about 30 minutes). In some embodiments, the shortened workflow does not include a library quantitation quality control (QC) steps (e.g., about 60 minutes). In some embodiments, a WTA step is directly followed by an indexing PCR reaction in the shortened workflow. Therefore, the shortened workflow (S3+ protocol) can take only about 5 hours and 15 minutes to complete.

Error Correcting Counts by Mapping Site

In some embodiments, a method of counting target molecules in a sample comprises the use of error correcting barcodes. In some embodiments, the transcript counts derived from mapping sites can be further refined by combining with a limited set of defined, error-correcting barcodes. The set of error-correcting barcodes can comprise about 10, about 20, about 30, about 40, about 50, about 60, about 70, about 80, about 90, about 100, or more than about 100 distinct error-correcting barcodes. The set of error-correcting barcodes can be added prior to amplification, or by performing reverse transcription or the second strand synthesis reactions under conditions with a high misincorporation rate (e.g., thereby incorporating random bases at a high per-base rate). The set of error-correcting barcodes can be used to non-uniquely tag the initial template nucleic acid molecules. In some embodiments, the set of error-correcting barcodes is too few to be used as UMI themselves, as many transcripts can be tagged with the same barcode. However, the error-correcting barcodes can be used to error correct counts elucidated from unique mapping sites, by imposing a requirement that sequencing reads must have the same mapping site and error correction barcode to be counted as being derived from the same original molecule.

The spectrum of random base changes which are incorporated under high mutation conditions can also be used to further error correct transcript counts generated based on the mapping sites (e.g., of truncation locations of aligned sequencing reads) alone, as all progeny of a given molecule can have the same or very similar unique mutation patterns. For example, such random base changes can be performed in either the reverse transcription or second strand synthesis steps, by randomly changing bases at a high rate, such that a contiguous n-base portion of a given molecule as compared to another identical molecule has a different set of base changes that occurred at different base locations of the molecules. The mutation profiles or “fingerprints” can be used in isolation to identify progeny polynucleotides from the same original nucleic acid molecule, by requiring that all sequencing reads derived from the same template nucleic acid molecule overlap to link mutation fingerprints derived from distant parts of the transcript.

In some embodiments, sequencing libraries can be generated such that the initial truncation site of the nucleic acid molecules is maintained. This can ensure the overlapping of sequencing reads, even at a relatively low sequencing coverage or depth, as each of the sequencing reads derived from a single nucleic acid molecule is located at the same site in the nucleic acid molecule. Though mapping sites may or may not be explicitly utilized and considered in the method for molecule counting, the generation of sequencing libraries where the initial truncation site is maintained can be crucial for enabling such molecule counting approaches at reasonable sequencing coverage or depth. Further, the identification or quantification of mapping sites can be used to improve the filtering of erroneous UMIs, by confirming that all UMIs which are being collapsed into the same individual molecule count all have the same mapping site as well.

Methods and systems of the present disclosure can leverage one or more improvements to enable robust molecular counting by mapping site, including: (1) methods for generating random truncations of transcripts or derived complementary DNA of a defined size prior to amplification, (2) methods for generating sequencing libraries from the truncation products that maintain the truncation site in an identifiable form in the final sequencing library, and (3) methods for counting molecules by utilizing read mapping sites to generate original transcript counts.

Fragmentation

Standard library preparation procedures for low RNA amounts can typically amplify the cDNA molecules prior to fragmentation, thereby losing the ability to maintain a single unique truncation site across all progeny polynucleotides of the original template nucleic acid molecule. For 3′-barcoded libraries, the fragmentation process can be used to retain the bead barcode information on the truncation. For example, this can be achieved by truncating the 5′ end of the nucleic acid molecule, or fragmenting the nucleic acid molecule before the 3′ barcode is linked to the nucleic acid molecule. Methods and systems of the present disclosure can comprise performing fragmentation of template nucleic acid molecules prior to amplification, such that the truncation site is maintained across all progeny polynucleotide molecules.

In some embodiments, the initial transcript or the first or second strand cDNA molecules can each be randomly cleaved. This random cleavage can be performed through a number of mechanisms, such as base-catalyzed hydrolysis, ultrasonic shearing, or partial enzymatic degradation. However, cleavage solutions can be challenging to implement on small amounts of input nucleic acid molecules without encountering undesirable loss of transcripts.

Alternatively, the reverse transcription product can be randomly truncated by spiking the reaction with a chain-terminating nucleotide, such as a dideoxynucleotide. For example, the concentration of the terminator which is added to the reverse transcription reaction can be tuned to create a desired distribution of truncation sizes of the fragments. In some embodiments, the chain terminating nucleotide can be chemically labeled on the 3′ carbon position to enable chemical ligation of the universal 5′ primer site (e.g., with or without error correction barcodes) to the truncated cDNA molecules. This can be performed using, for example, a Click chemistry or other chemistries.

In some embodiments, during second strand synthesis, a plurality of second strand truncated nucleic acid molecules are formed. In some embodiments, the second strand nucleic acid molecules are truncated randomly. In some embodiments, during second stand synthesis, random truncations can be generated by priming the extension with a tailed randomer. The tailed randomer can typically be a random polynucleotide (e.g., having 9 or 10 bases), which is linked on its 5′ side to a universal primer sequence (UPS), either with or without an error correction barcode. The primer concentrations, hybridization conditions, and extension conditions can be tuned to create a desired distribution of truncation sizes of the fragments. In some embodiments, random transposon insertion can be performed to randomly fragment nucleic acid molecules after the second strand synthesis has been performed.

Sequence Library Generation

After fragmentation, the truncated molecules can be amplified and directionally tagged with adaptor sequences to create the final sequence libraries. Typically, optimal amplification of sequencing libraries derived from a limited amount of starting material (e.g., a relatively small number of template nucleic acid molecules) can require the use of suppression PCR amplification. The suppression PCR amplification can utilize the same universal priming sequence (UPS) on both sides of the amplicon to inhibit the amplification of primer dimers and other small products, through the formation of a hairpin structure which is nucleated by the intramolecular binding of the two primer sites, thereby inhibiting the binding of the amplification primer. However, using suppression PCR to generate sequencing libraries with no further truncation of the transcript-derived sequence can encounter challenges due to a need to re-establish the directionality of the sequencing library. For example, the first sequencing read can be required to capture the 3′ barcode on each sequencing read.

In some embodiments, the re-establishment of directionality is achieved by including sided sequences (SS) on the 3′ side of the universal primer site (UPS) on the 3′ and/or 5′ sides of the sequencing libraries. SS can be configured to be included in read 1 and/or read 2 during sequencing and thus enabling the identification of the directionality of the resultant sequencing library, thereby identifying the truncation mapping sites. The SS can be a known sequence. The SS can be a designed sequence. In some embodiments, the size of the SS can be limited to 2 to 5 bases. In some embodiments, the SS has a length of about 2-16 bases, such as 2, 3, 4, 5, 6, 7, 8, 9, 10, 11, 12, 13, 14, 15 or 16 bases. In some embodiments, the size of the SS is about 16-32 bases. In some embodiments, the SS has a length of about 7 bases. In some embodiments, the SS has a length of 5, 6, 7, 8, or 9 bases. In some embodiments, the SS has a length of 5-9 bases. In some embodiments, the SS has a length of 2-10 bases. In some embodiments, the SS has a length of 6-12 bases. In some embodiments, additional sequence length which is added to the second strand synthesis primer results in a decreased priming efficiency and therefore complexity of the sequencing library. The final sequencing library can be created by amplifying the suppression PCR product with indexing primers which contain (in the 5′-3′ direction) an adaptor sequence for the sequencing platform, an index sequence for indexing of the sequencing library, and a custom sequencing primer sequence. The custom sequencing primer sequence can include, on its 3′ end, a portion of the UPS sequence and the SS sequence that defines the 3′ or 5′ side of the sequencing library. Though the primers have nearly the same binding affinity to each side of the product, primer extension can only occur if the primer is bound to the correct side, since mismatches between template and primer at the 3′ can significantly disrupt primer extension. This selection can be further enhanced by connecting the SS bases to the primer (e.g., using thiophosphate bonds), thereby preventing removal of the bases by exonuclease activity of the polymerase. In some embodiments, the UPS sequence is required to be long enough to facilitate binding of the primer to the suppression PCR product in concert with the SS sequence, but short enough such that correct matching of the SS sequence biases the primer to bind the correct side and the two sequencing primer sequences are differentiated to a sufficient extent to prevent hairpin formation on the sequencer flowcell.

In some embodiments, a method described herein comprises a PCR annealing step and the length of the step is extended from about 30 seconds up to about five minutes. This can improve sequencing library yields in an incremental manner, possibly by enabling multiple rounds of binding and melting of the primer, thereby increasing the chances that a polymerase encounters the correct primer bound to each site to initiate extension.

In some embodiments, to improve the total workflow time, a second PCR reaction (indexing reaction) can be performed without requiring purification of the suppression PCR product. The second PCR reaction can add adaptor and index sequences to the amplification product, while preserving the truncation mapping sites. In some embodiments, the primers of the second PCR reaction are specific for the 5′ and/or 3′ sided sequence with 5′ tails containing the appropriate adaptor. The method can comprise transferring a portion of the reaction to a new PCR tube, and adding a 1×PCR master mix containing the indexing primers, a DNA polymerase and, a single-strand specific exonuclease. The indexing primers can be protected from degradation on both sides by phosphothioate bonds. The reaction can be performed using a thermocycler, and an initial 5-minute, 37° C. incubation can be performed to allow the exonuclease to degrade the remaining suppression PCR primers. Next, the adaptor sequences can be added using the index primers by performing 5 cycles of 95° C. for 30 sec., 60° C. for 5 min, and 72° C. for 30 sec. Next, after completion of the thermal cycling, a 5′-3′ ds-DNA exonuclease and 3′-5′ single strand-specific exonuclease can be added to degrade DNA molecules that do not contain the index primer sequence. Next, the remaining DNA molecules can be purified and quantitated for sequencing.

In some embodiments, a similar selection process can be performed for nucleic acid molecules extended in the second reaction. For example, this can be achieved by incorporating deoxyuracil bases during the initial suppression PCR reaction, and then degrading all molecules containing uracil after the second reaction (e.g., using uracil DNA glycosylase and endonuclease VIII).

In some embodiments, molecule counting based on mapping sites can be performed using a bioinformatics pipeline in which all reads with the same sample barcode and genomic mapping site are attributed to the same original template nucleic acid molecule, and therefore are collapsed into the same molecule count. The method can comprise sequencing compatible sequencing libraries (e.g., by paired-end sequencing). The sample barcode can captured in the first sequencing read of the sequencing read pair, and the transcript sequence can be captured in the second sequencing read of the sequencing read pair. The second sequencing read can be aligned to a defined genome, and the specific mapping location (e.g., location of the genome to which the second sequencing read aligns) can be identified and/or quantified. The sample barcode for each read pair can be identified and/or quantified from the first sequencing read of the sequencing read pair. All sequencing read pairs that have the same sample barcode and the same mapping site can be attributed to the same original template nucleic acid molecule and therefore collapsed into a single molecule count.

In some embodiments, if error correcting barcodes are also included in the sequencing library creation, the error correcting barcodes identified and/or quantified from the second sequencing read of the sequencing read pair. Only sequencing reads sharing the same sample barcode, the same mapping site, and the same error correcting barcode can be attributed to the same original template nucleic acid molecule and therefore are collapsed into a single molecule count. After the sequencing reads are collapsed, the number of counts mapping to each gene are counted to yield the final transcript count for each gene in each sample.

### Methods for Depleting or Enriching Target Sequences in Sequencing Libraries

Unbiased profiling of transcripts can be a powerful tool for understanding the biology of a sample. However, to save on sequencing costs and to acquire specific information about the sample, it is often desirable to deplete over-represented sequences, such as ribosomal RNAs and target-specific sequences within a given transcript, such as variable regions in T-cell and B-cell receptor transcripts or specific single nucleotide polymorphisms (SNPs) or splicing junctions.

The present disclosure provides methods for depleting or enriching specific sequences in the context of an otherwise unbiased sequencing library preparation, using second strand synthesis primed with a tailed-randomer primer. The methods for enriching or depleting specific sequences in a sequencing library can comprise including in the second strand synthesis reaction a set of 3′-blocking oligonucleotides that are identical to (or are otherwise a copies of) unwanted sequences. The set of blocking oligonucleotides can have an annealing temperature higher than the randomer primer. An annealing step can be performed at a temperature such that the set of blocking oligonucleotides bind but the randomer does not. This can ensure that the blocking oligonucleotides blanket the undesired sequence before the tailed-randomer primer can bind. This approach can be leveraged to deplete one or more specific transcripts from a sequencing library, or to ensure that one or more specific portions of a transcript are present in a sequencing library, and to define the exact location of the sequencing read in specific transcripts, while all other untargeted sequences are being captured in an unbiased manner. The set of blocking nucleotides can each have a length of about 5, about 10, about 15, about 20, about 25, about 30, about 35, about 40, about 45, about 50, about 55, about 60, about 65, about 70, about 75, about 80, about 85, about 90, about 95, about 100, or more than about 100 bases. In some embodiments, the set of blocking oligonucleotides comprise oligonucleotides having from about 5 to about 200 bases, from about 10 to about 150 bases, from about 15 to about 100 bases, from about 20 to about 75 bases, from about 25 to about 50 bases, and/or ranges therebetween. In some embodiments, the set of blocking oligonucleotides comprise oligonucleotides with at least 5 bases, at least 10 bases, at least 20 bases, at least 30 bases, at least 40 bases, at least 50 bases, or at least 75 bases. In some embodiments, the set of blocking oligonucleotides comprise oligonucleotides with at most 10 bases, at most 20 bases, at most 30 bases, at most 40 bases, at most 50 bases, at most 75 bases, at most 100 bases, or at most 150 bases. In some embodiments, each of said set of blocking oligonucleotides comprises from about 20 to about 100 bases, and/or ranges therebetween.

In an aspect, the present disclosure provides a method for depleting a sample for one or more target sequences. In some embodiments, the method comprises obtaining a sample comprising a plurality of template nucleic acid molecules, wherein said template nucleic acid molecules comprise one or more target sequences or copies thereof. In some embodiments, the method comprises combining said plurality of template nucleic acid molecules with a set of blocking oligonucleotides. In some embodiments, the set of blocking oligonucleotides is configured to bind with at least one of said one or more target sequences. The blocking oligos can have an annealing temperature higher than the randomer that is present in the second strand synthesis primer. In some embodiments, the method comprises annealing at least one of said one or more target sequences with at least one of said set of blocking oligonucleotides. In some embodiments, an annealing step at a temperature where the blocking oligos bind but the randomer does not can be added to ensure the blocking oligos blanket the undesired sequence before the randomer can bind. In some embodiments, the entire template nucleic acid molecule is blocked and a second strand primer does not hybridize to the blocked template nucleic acid. In some embodiments, the method comprises contacting said plurality of template nucleic acid molecules with a plurality of second strand primers. The plurality of second strand primers comprise a 5′ universal primer sequence and a 3′ sequence complementary to a sequence of said template nucleic acid. In some embodiments, the method comprises extending said plurality of second strand primers to produce a plurality of second strand nucleic acid molecules. In some embodiments, the method comprises one or more steps selected from: (a) obtaining a sample comprising a plurality of template nucleic acid molecules, wherein said template nucleic acid molecules comprise one or more target sequences; (b) combining said plurality of template nucleic acid molecules with a set of blocking oligonucleotides, wherein said each of said set of blocking oligonucleotides is configured to bind with at least one of said one or more target sequences, thereby annealing at least one of said one or more target sequences with at least one of said set of blocking oligonucleotides; (c) contacting said plurality of template nucleic acid molecules with a plurality of second strand primers, wherein each of said plurality of second strand primers comprises a 5′ universal primer sequence and a 3′ sequence complementary to a sequence of said template nucleic acid; and (d) extending said plurality of second strand primers to produce a plurality of second strand nucleic acid molecules, thereby depleting at least one of said one or more target sequences. In some embodiments, the method further comprises quantifying the target sequences before and/or after the depletion step. In some embodiments, the method further comprises sequencing the target sequences before and/or after the depletion step. In some embodiments, the target sequence is reduced to at most 90% relative to its content before the enrichment. In some embodiments, the target sequence is reduced to at most 50%, 40%, 30%, 20%, 10%, 5%, 2%, 1%, or less than 1% relative to its content before the enrichment.

In another aspect, the present disclosure provides a method for enriching a sample for one or more target sequences. In some embodiments, the method comprises obtaining a sample comprising a plurality of template nucleic acid molecules, wherein said template nucleic acid molecules comprise one or more target sequences or copies thereof. In some embodiments, the method comprises combining said plurality of template nucleic acid molecules with a set of blocking oligonucleotides. In some embodiments, the set of blocking oligonucleotides comprises a sequence complementary to a template nucleic sequence that is 3′ to one of said target sequences. In some embodiments, the method comprises annealing at least one of said set of blocking oligonucleotides to said template nucleic acid sequence that is 3′ to one of said target sequences. In some embodiments, the method comprises contacting said plurality of template nucleic acid molecules with a plurality of second strand primers. The plurality of second strand primers can comprise a 5′ universal primer sequence and a 3′ sequence complementary to a sequence of said template nucleic acid. In some embodiments, the method comprises extending said second strand primers to produce a plurality of second strand nucleic acid molecules, thereby enriching at least one of said one or more target sequences. The extension of the second strand primers can displace the blocking oligos. In some embodiments, the method comprises one or more steps selected from: (a) obtaining a sample comprising a plurality of template nucleic acid molecules, wherein said template nucleic acid molecules comprise one or more target sequences; (b) combining said plurality of template nucleic acid molecules with a set of blocking oligonucleotides, wherein said set of blocking oligonucleotides comprises a sequence complementary to a template nucleic sequence that is 3′ to one of said target sequences, thereby annealing said template nucleic acid sequence that is 3′ to one of said target sequences with at least one of said set of blocking oligonucleotides; (c) contacting said plurality of template nucleic acid molecules with a plurality of second strand primers, wherein each of said plurality of second strand primers comprises a 5′ universal primer sequence and a 3′ sequence complementary to a sequence of said template nucleic acid; and (d) extending said second strand primers to produce a plurality of second strand nucleic acid molecules, thereby enriching at least one of said one or more target sequences. In some embodiments, the 3′ sequence complementary to a sequence of said template nucleic acid comprises a random sequence. In some embodiments, the 3′ sequence complementary to a sequence of said template nucleic acid is complementary to a template nucleic sequence 5′ to one of said target sequences. In some embodiments, the method further comprises quantifying the target sequences before and/or after enrichment. In some embodiments, the method further comprises sequencing the target sequences before and/or after enrichment. In some embodiments, the target sequence is enriched at least 2 fold relative to its content before the enrichment. In some embodiments, the target sequence is enriched at least 10, 10², 10³, 10⁴, or 10⁵ relative to its content before the enrichment.

In some embodiments, the 3′ sequence of the second strand primer has a first annealing temperature, and the set of blocking oligonucleotides hybridize to the template nucleic acids at a second annealing temperature. In some embodiment, the first annealing temperature is higher than the second annealing temperature. In some embodiment, the first annealing temperature is lower than the second annealing temperature. In some embodiment, the first annealing temperature is about the same as the second annealing temperature. In some embodiments, the method comprises contacting the plurality of template nucleic acid molecules with the plurality of second strand primers at a third annealing temperature. In some embodiments, the third annealing temperature is about the same as the second annealing temperature. In some embodiments, the third annealing temperature is lower than the second annealing temperature. In some embodiments, the third annealing temperature is about the same as the first annealing temperature. In some embodiments, the third annealing temperature is higher than the first annealing temperature. In some embodiments, the third annealing temperature is greater than the first annealing temperature and less than said second annealing temperature.

The method for depleting specific sequences in a sequencing library can comprise complete depletion of a transcript, such as an rRNA molecule. For example, a set of blocking oligonucleotides covering the entire target transcript sequence can be added to prevent the tailed randomer primer from binding anywhere on the transcript, thereby preventing linking of the 5′ universal primer sequence (UPS) required for amplification of the nucleic acid molecule. Blocking oligonucleotides which are designed to fully block one or more specific transcripts can be typically longer (e.g., about 20 to 50 bases) to ensure they do not melt during the second strand synthesis reaction.

The method for enriching specific sequences in a sequencing library can be performed to ensure inclusion of specific portions of a transcript in the final sequencing library. This can be achieved by adding a set of blocking oligonucleotides which are identical and/or complementary to the undesired sequence and all sequences 3′ to the desired sequence in the transcript. During annealing in the second strand synthesis reaction, the set of blocking oligonucleotides can bind the complementary sequences in the first strand cDNA molecule, thereby preventing the tailed-randomer primer from binding in this region and ensuring that it binds upstream of the target region. In a herein described sequencing libraries, second strand cDNA molecules primed by the tailed-randomer primer can be extended through the blocked region to acquire the 3′ barcode and the 3′ universal primer sequence (UPS). This can be achieved through several mechanisms.

As one example, a two-step extension reaction which contains both a mesophilic and thermophilic DNA polymerase can be performed. The extension can be initiated at a lower temperature (37° C.) to extend as far as possible the tailed-randomer primer on all transcripts. This can be followed by an extension time at elevated temperature (e.g., about 60° C. to about 72° C.), such that the blocking oligonucleotides on specific transcripts can melt from the first strand cDNA. The stalled randomer product can then be extended through the blocked region.

As another example, a polymerase with high strand displacement activity can be leveraged in the second strand synthesis reaction to displace the blocking oligonucleotides when they are encountered.

As another example, separate annealing and extension steps can be performed. The set of blocking oligonucleotides can include bases, such as deoxyuracil, that induce cleavage by specific enzymes, such as uracil nucleotide glycosylase. Both the blocking oligonucleotides and the tailed-randomers primers can be annealed in a single step and then washed away. The bound oligonucleotides can then be extended in a reaction mix that contains a DNA polymerase and the blocking oligonucleotide cleaving enzyme. The blocking oligonucleotide cleavage can be performed in a separate step in between annealing and extension, to ensure complete cleavage prior to extension.

Further, other approaches for removing the blocking oligonucleotides during second strand synthesis can be performed, such as using DNA polymerases with 5′-3′ exonuclease activity to degrade the blocking oligonucleotides.

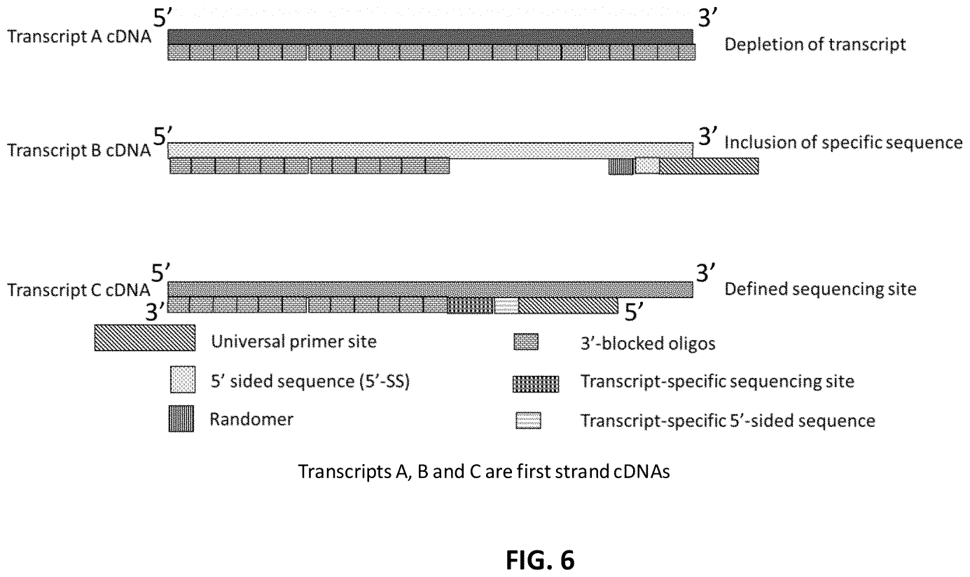

FIG. 6 shows a schematic depicting depletion or enrichment of specific transcript sequences in a final sequencing library, by leveraging blocking oligonucleotides during second strand synthesis, in accordance with disclosed embodiments.

As an example, to deplete a transcript A cDNA molecule, a set of blocking oligonucleotides which are complementary to the entire first strand cDNA sequence is added. The blocking oligonucleotides prevent the random second strand synthesis primer from binding anywhere on the transcript, thereby preventing the amplification of transcript A in the following PCR step.

As another example, to ensure that a specific portion of a transcript B cDNA molecule is included in the sequencing library, blocking oligonucleotides complementary to the region which is 3′ to the region of interest can be included, to prevent the random second strand synthesis primer from binding in this region. The region of interest can then be copied and included in a second strand cDNA when a second strand synthesis primer attaches to a position 5′ to the region and extends to generate the second strand cDNA. The second strand synthesis primer used in this method can comprise universal primer sequence, a sided sequence (5′ SS), and/or a randomer that is configured to attached to the first stand cDNA.

In some embodiments, a method of including a region/sequence of interest comprises attaching blocking oligos to a region that is 3's to the region of interest to prevent priming in this region. In some embodiments, the method comprises attaching a second strand synthesis primer to a position that is 5′ to the region/sequence of interest. In some embodiments, the method comprises extending a second strand synthesis primer that comprises a universal primer sequence, optionally a sided sequence (5′ SS), and a randomer. During second strand synthesis, the polymerase extends from the randomer and displaces the blocking oligos, thereby generating a second strand cDNA that comprises a copy of the sequence of interest.

In some embodiments, a method of including a specific or target region/sequence of interest comprises attaching blocking oligos to a region that is 3's to the specific region of interest. In some embodiments, the method comprises extending a second strand synthesis primer that comprises a universal primer sequence, optionally a region-specific sided sequence (5′ SS), and a sequence that is configured to specifically attach to the region of interest, thereby generating a second strand cDNA that comprises a copy of the specific sequence of interest.

As another example, to define the specific sequencing read start site of a transcript C cDNA molecule that is to be sequenced in the standard sequencing reaction, a primer that is specific for the region which is just 5′ to the desired sequencing start site and that has the same 5′ universal primer sequence (UPS) tail as the random primer is included in the second strand synthesis reaction. The site-specific primer can also comprise a region/site specific sided sequence.

The method for enriching specific sequences in a sequencing library can comprise defining the exact location of the sequencing read in the final sequencing library. This can be achieved by including in the reaction a primer with a 3′ sequence which is identical to (or is otherwise a copy of) a location of a desired starting position of the sequencing read, linked to the 5′ universal primer sequence (UPS). In some embodiments, blocking oligonucleotides that are identical to all sequences which are complementary to the sequencing site are also included. During second strand synthesis, the specific primer can be extended through the blocked sequence, such as using one of the approaches outlined above, to yield a sequencing library molecule with a defined sequencing start location for the particular transcript. Any one or more of the above approaches can be performed in the same reaction on multiple transcripts.

In some embodiments, a unique SS sequence is included in the primer to enable amplification of only the nucleic acid molecules that are extended in this fashion. This can be useful if longer sequencing reads are needed to extend through the targeted region compared to the rest of the unbiased library, such as the case with variable regions in T-cell and B-cell receptor transcripts.

### Constructing Sequencing Libraries

In another aspect, the present disclosure provides a method for constructing a sequence library for sequencing a plurality of template nucleic acid molecules. In some embodiments, a sequence library described herein comprises truncation mapping site information, thus enabling molecule counting of the template and/or target nucleic acids based on the unique truncation sites. In some embodiments, a sequence library described herein comprises directionality information of the nucleic acids. In some embodiments, the method comprises contacting a plurality of template nucleic acid molecules with a plurality of second strand primers. The plurality of second strand primers can comprise a 5′ universal primer sequence and a 3′ sequence complementary to a sequence of said template nucleic acid molecules. In some embodiments, the method comprises extending said plurality of second strand primers to produce a plurality of second strand nucleic acid molecules. In some embodiments, the method comprises amplifying said plurality of second strand nucleic acid molecules with a plurality of indexing primers. The plurality of indexing primers can comprise (e.g., in a 5′-3′ direction) an adaptor sequence, an index sequence for indexing of said sequencing library, and a custom sequencing primer sequence. In some embodiments, the method comprises one or more steps selected from: (a) contacting a plurality of template nucleic acid molecules with a plurality of second strand primers, wherein each of said plurality of second strand primers comprises a 5′ universal primer sequence and a 3′ sequence complementary to a sequence of said template nucleic acid molecules; (b) extending said plurality of second strand primers to produce a plurality of second strand nucleic acid molecules; and (c) amplifying said plurality of second strand nucleic acid molecules from (b) with a plurality of indexing primers, wherein said plurality of indexing primers comprise, in a 5′-3′ direction, an adaptor sequence, an index sequence for indexing of said sequencing library, and a custom sequencing primer sequence. In some embodiments, the 3′ sequence of the second strand primer hybridizes with said template nucleic acid in a site-nonspecific fashion. In some embodiments, the 3′ sequence comprises a random sequence. In some embodiments, the 3′ sequence comprises a semi-random sequence. In some embodiments, the 3′ sequence comprises a specific sequence that hybridizes with a sequence of interest in the template nucleic acid.

In another aspect, the present disclosure provides a system comprising one or more selected from: (a) a plurality of beads; (b) a plurality of cDNA molecules, wherein each of said plurality of beads comprises a first strand of a cDNA molecule of said plurality of cDNA molecules attached thereto; and (c) a plurality of second strand primers for performing second strand synthesis of said plurality of cDNA molecules to produce a sequencing library, wherein each of said plurality of second strand primers comprises a 5′ universal primer sequence, a 3′ sequence complementary to a sequence of said first strand cDNA, and a knownsided sequence (SS) of 2-5 bases. In some embodiments, the plurality of second strand primers is configured to produce a truncation site of a second strand of a cDNA molecule of said plurality of cDNA molecules during said second strand synthesis. In some embodiments, the second strand primers produce random truncations sites from the first strand cDNA molecules.

In another aspect, the present disclosure provides a system comprising one or more selected from: (a) a plurality of second strand primers for performing second strand synthesis of a plurality of cDNA molecules to produce a sequencing library, wherein each of said plurality of second strand primers comprises a 5′ universal primer sequence, a 3′ random template nucleic acid-binding sequence, and a sided sequence (SS), wherein said plurality of second strand primers is configured to produce a truncation site of a second strand of a cDNA molecule of said plurality of cDNA molecules during said second strand synthesis; and (b) a plurality of indexing primers comprising (e.g., in a 5′-3′ direction) an adaptor sequence, an index sequence for indexing nucleic acid molecules of said sequencing library, and known-sided sequences (SS) that define a 3′ or a 5′ side of said nucleic acid molecules of said sequencing library.

### Methods and System

In one aspect, disclosed herein are methods of detecting or monitoring a disease or condition in a subject. In some embodiments, the method comprises counting nucleic acid molecules of a sample according to a method as described herein, wherein said sample is a biological sample obtained from said subject. The number of target nucleic acid molecules (e.g., RNAs) can be associated with said disease or condition. In some embodiments, the disease or condition is a proliferative disease, an autoimmune disease, or an infectious disease. In one aspect, disclosed herein are methods of assaying a sample bioparticle, comprising counting nucleic acid molecules of a sample according to a method as described herein, wherein said sample is obtained from a bioparticle and wherein said bioparticle is a T cell or a B cell. In some embodiments, the method comprises releasing RNA molecules from said cell or bioparticle. In some embodiments, the method comprises performing reverse transcription reaction of said RNA molecules thereby forming said plurality of template nucleic acid molecules. In some embodiments, the bioparticle is obtained from a subject.

In one aspect, disclosed herein are methods of detecting or monitoring a disease or condition in a subject. In some embodiments, the method comprises one or more steps selected from: obtaining a sample fluid from a subject, wherein said sample fluid comprises a plurality of bioparticles; loading said sample fluid onto a microwell array that comprises a plurality microwells, thereby loading a bioparticle into at least one microwell; releasing one or more target nucleic acid molecules (e.g., RNAs) from said bioparticle; producing template nucleic acid molecules, each comprising a copy of a sequence of said target nucleic acid molecules; and identifying a number of target nucleic acid molecules present in said bioparticle. In some embodiments, the method comprises randomly truncating the template nucleic acid molecules at a truncation base position within said template nucleic acid molecules. In some embodiments, the truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position. In some embodiments, the method comprises, amplifying at least a portion of said plurality of truncated nucleic acid molecules to produce a plurality of amplified nucleic acid molecules, wherein said truncation base positions are preserved in said plurality of amplified nucleic acid molecules. In some embodiments, the method comprises sequencing at least a portion of said amplified nucleic acid molecules or said truncated nucleic acid molecules to determine a number of unique truncation base positions. In some embodiments, the method comprises identifying a number of target nucleic acid molecules present in said bioparticle using said number of unique truncation base positions. In some embodiments, the sample fluid comprises a bodily fluid of said subject. In some embodiments, the sample fluid comprises a blood sample of said subject. In some embodiments, the plurality of bioparticles comprise peripheral blood mononuclear cells (PBMCs). In some embodiments, the plurality of bioparticles comprise engineered cells. In some embodiments, the plurality of bioparticles comprise immune cells. In some embodiments, the plurality of bioparticles comprise T cells. In some embodiments, the T cells comprise native T cells, engineered T cells, or both. In some embodiments, the T cells comprise one or more native T cells and one or more chimeric antigen receptor (CAR)-T cells. In some embodiments, the method comprises, after loading said sample fluid, storing said microwell array comprising said bioparticle in said at least one microwell for a period of time. In some embodiments, the period of time is between 1 hour and 30 years, and/or ranges therebetween as described elsewhere herein.

The described methods can also be used to determine and correlate the clonal lineage of engineered cells. In some embodiments, the describe methods are used to perform quality control in the manufacturing of engineered cells. In some embodiments, the target and/or template nucleic acid molecules are indicative of clonal lineage of said engineered cells. For example, in some embodiments, the method comprises identifying and comparing the number of target and/or template nucleic acid molecules of cells obtained from a subject at the same or different time. For example, in some embodiments, the method comprises identifying and comparing the number of target and/or template nucleic acid molecules of a cell obtained from a subject and an in vitro cell. In some embodiments, the method comprises identifying and comparing the number of target and/or template nucleic acid molecules of an engineered cell obtained from a subject and an in intro engineered cell. In some embodiments, the cell obtained from the subject, the in intro cell, or both have been independently stored in the microwell for a period time. In some embodiments, the engineered cell are edited by clustered regularly interspaced short palindromic repeats (CRISPR) associated proteins. In some embodiments, the target nucleic acid molecules comprises a sequence of a guide RNA. In some embodiments, the template nucleic acid molecule is a cDNA. In some embodiments, the target and/or template nucleic acid molecules encode a sequence of a CRISPR associated protein.

In one aspect, disclosed herein are methods of assaying a plurality of engineered cells, comprising one or more steps selected from: obtaining a sample fluid comprising a plurality of engineered cells; loading said sample fluid onto a microwell array that comprises a plurality of microwells, thereby loading an engineered cell into one microwell; releasing one or more target nucleic acid molecules from said engineered cell; and identifying a number of target nucleic acid molecules present in said engineered cell. In some embodiments, the method comprises producing template nucleic acid molecules, each comprising a copy of a sequence of said target nucleic acid molecules. In some embodiments, the method comprises randomly truncating said template nucleic acid molecules at a truncation base position within said template nucleic acid molecules. In some embodiments, the truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position. In some embodiments, the method comprises amplifying at least a portion of said plurality of truncated nucleic acid molecules to produce a plurality of amplified nucleic acid molecules, wherein said truncation base positions are preserved in said plurality of amplified nucleic acid molecules. In some embodiments, the method comprises sequencing at least a portion of said amplified nucleic acid molecules or said truncated nucleic acid molecules to determine a number of unique truncation base positions. In some embodiments, the method comprises identifying a number of template nucleic acid molecules present in said engineered cell using said number of unique truncation base positions. In some embodiments, the engineered cells comprise exogenous nucleic acid sequences. In some embodiments, the target and/or template nucleic acid molecules comprise the exogenous nucleic acid sequences. In some embodiments, the target and/or template nucleic acid molecules comprise native sequences. In some embodiments, the target and/or template nucleic acid molecules lack exogenous nucleic acid sequences. In some embodiments, the engineered cells lack one or more knock-out sequences. In some embodiments, the target and/or template nucleic acid molecules lack said knock-out sequences. In some embodiments, the target and/or template nucleic acid molecules comprise said knock-out sequences. In some embodiments, the method comprises, after loading said sample fluid, storing said microwell array comprising said engineered cell in said at least one microwell for a period of time. In some embodiments, the period of time is between 1 hour and 30 years, and/or ranges therebetween as described elsewhere herein.

In some embodiments, the engineered cells comprise engineered immune cells. In some embodiments, the engineered cells comprise engineered stem cells. In some embodiments, the engineered cells are engineered immune cells such as T cells, B cells, NK cells, bone marrow cells, plasma cells, immunoglobulins, neutrophils, monocytes, red blood cells, and dendritic cells. In some embodiments, the engineered cells comprise engineered T cells, engineered B cells, or a combination thereof. In some embodiments, the engineered cells comprise engineered secreting cells such as protein-secreting cells. For example, in some embodiments, the engineered cells are insulin-secreting cells. In some embodiments, the engineered cells are γ-aminobutyric acid (GABA)-secreting cells.

In some embodiments, engineered cells described herein comprise chimeric antigen receptor (CAR)-T cells. In some embodiments, the target nucleic acid molecules comprise RNA molecules of said engineered cell. In some embodiments, the target nucleic acid molecules comprise DNA molecules of said engineered cell. In some embodiments, the template nucleic acid molecules comprise cDNA molecules of said engineered cell. In some embodiments, the target and/or template nucleic acid molecules encode a sequence of an immune receptor that is a T-cell receptor (TCR), a B-cell receptor (BCR), a cytokine receptor, a chemokine receptor, a major histocompatibility complex (MHC) class I molecule, a MHC class II molecule, a Toll-like receptor, a killer activation receptor (KAR), a killer-cell immunoglobulin-like receptor (KTR), or an integrin. In some embodiments, the target and/or template nucleic acid molecules encode a sequence of a TCR. In some embodiments, the target and/or template nucleic acid molecules encode a sequence of a complementarity determining region (CDR) from T-cell receptor genes or immunoglobulin genes. In some embodiments, the CDR comprises CDR1, CDR2, or CDR3. In some embodiments, the target and/or template nucleic acid molecules encode a sequence of a protein secreted by T cells. In some embodiments, the target and/or template nucleic acid molecules are indicative of clonal lineage of said engineered cells. In some embodiments, the bioparticle is a chimeric antigen receptor (CAR)-T cell. In some embodiments, the target and/or template nucleic acid molecules comprise sequences of a complementarity determining region (CDR) from T-cell receptor genes. In some embodiments, the target and/or template nucleic acid molecules are indicative of contamination of said CAR-T cell. In some embodiments, the target and/or template nucleic acid molecules are indicative of clonal lineage of said CAR-T cell.

The analysis of T cell or B cell receptors can comprise the enrichment of the receptors. In some embodiments, a method of assaying T cells or B cells comprises enriching a sequence that encodes a portion of the corresponding CDR region. The enrichment can comprise a procedure or steps described in the present disclosure. The enrichment can also use a method known in the art, e.g., methods disclosed in WO 2018/132635 A1, which is hereby incorporated by reference in its entirety.

In one aspect, disclosed herein are methods of assaying engineered cells. In some embodiments, the method comprises detecting, verifying the presence, or counting the number of an exogenous nucleic acid sequence of said engineered cell. In some embodiments, the method comprises (a) obtaining a sample fluid comprising a plurality of engineered cells, wherein said plurality of engineered cells comprise exogenous genes; (b) loading said sample fluid onto a microwell array that comprises a plurality of microwells, thereby loading an engineered cell into one microwell; and (c) releasing one or more target nucleic acid molecules from said engineered cell, wherein said target nucleic acid molecules comprise one or more said exogenous genes. In some embodiments, the method comprises detecting or counting a number of a nucleic acid sequence of an engineered cell, thereby verifying a gene knock-out. In some embodiments, the method comprises (a) obtaining a sample fluid comprising a plurality of engineered cells, wherein at least one of said plurality of engineered cells lacks a knock-out sequence; (b) loading said sample fluid onto a microwell array that comprises a plurality of microwells, thereby loading an engineered cell into one microwell; and (c) releasing one or more target nucleic acid molecules from said engineered cell. In some embodiments, the method comprises producing template nucleic acid molecules, each comprising a copy of a sequence of said target nucleic acid molecules. In some embodiments, the target and/or template nucleic acid molecules lack said knock-out sequence. In some embodiments, the target and/or template nucleic acid molecules comprise said knock-out sequence. In some embodiments, the method comprises one or more steps selected from: (d) randomly truncating said template nucleic acid molecules at a truncation base position within said template nucleic acid molecules, wherein said truncating comprises performing a random selection of said truncation base position among a plurality of base positions of said template nucleic acid molecules and making a copy of at least a portion of said template nucleic acid molecules, thereby producing a plurality of truncated nucleic acid molecules, wherein said plurality of truncated nucleic acid molecules preserve said truncation bases position; (e) optionally amplifying at least a portion of said plurality of truncated nucleic acid molecules to produce a plurality of amplified nucleic acid molecules, wherein said truncation base positions are preserved in said plurality of amplified nucleic acid molecules; (f) sequencing at least a portion of said amplified nucleic acid molecules or said truncated nucleic acid molecules to determine a number of unique truncation base positions; and (g) identifying a number of target and/or template nucleic acid molecules present in said engineered cell using said number of unique truncation base positions.

Methods and systems of the present disclosure can use microwell arrays to partition samples (e.g., single cells among a plurality of cells in a sample). Droplets based systems can also be used in the disclosed methods and systems.

A microwell array can comprise a plurality of microwells. In some cases, the microwell array comprises from about 1000 to about 1,000,000 microwells. In some cases, the microwell array comprises from about 5000 to about 1,000,000 microwells. In some cases, the microwell array comprises from about 50,000 to about 150,000 microwells. In some specific embodiments, the microwell array comprises about 50,000, about 55,000, about 60,000, about 65,000, about 70,000, about 75,000, about 80,000, about 85,000, about 90,000, about 95,000, about 100,000, about 105,000, about 110,000, about 115,000, about 120,0000, about 130,000, about 140,000, or about 50,000 microwells. The microwells can be arranged in any pattern. In some embodiments, the microwells are arranged in a hexagonal pattern.

A microwell can have a volume in the picoliter range, including volumes ranging from less than 1 picoliter to about 10,000 picoliters. The range can be from about 1 picoliter to about 1000 picoliters, or about 5 picoliters to about 1000 picoliters, or about 10 picoliters to about 500 picoliters, or about 50 picoliters to about 125 picoliters. A microwell can have dimensions (e.g., x and y or diameter, and height dimensions) in the micron ranges. For example, a microwell can have dimensions of about 45 microns (x) by about 45 microns (y) by about 60 microns (h) and have a rectangular volume, or they can have dimensions of about 50 microns (x) by about 50 microns (y) by about 50 (h) microns and have a cube volume. The microwell can have cross-sectional area (from a top-down perspective) that is square, hexagon, circular, oval, etc.

The microwell array can comprise a top surface, where the openings of the microwells are located. In some embodiments, an average diameter of the microwells on the top surface is at most 1000 microns, at most 500 microns, at most 400 microns, at most 300 microns, at most 200 microns, at most 100 microns, at most 75 microns, at most 50 microns, at most 40 microns, at most 30 microns, at most 20 microns, at most 10 microns, or at most 5 microns. In some embodiments, an average diameter of the microwells on the top surface is at least 5 microns, at least 7 microns, at least 10 microns, at least 20 microns, at least 30 microns, at least 45 microns, at least 50 microns, or at least 100 microns. In some embodiments, an average diameter of the microwells on the top surface is from about 5 microns to about 50 microns. In some embodiments, a microwell is configured to hold an object of interest, e.g., a bead, a cell, a fragment of a tissue, etc.

The microwells can comprise any suitable shape and geometry; for example, they can be cylindrical, cuboid, conical, etc. In some cases, the microwells comprise a uniform depth in a range of 5 microns to 500 microns. In some cases, the microwells are cylindrical and have a uniform diameter in a range of 1 micron to 500 microns (e.g., 15-100 microns or 1-10 microns). In some cases, the microwells are cuboid and have a uniform largest lateral length in a range of 1 micron-500 microns (e.g., 15-100 microns or 1-10 microns). In some cases, the microwells are conical and have a uniform diameter in a range of 35 microns to 100 microns at a top surface and can have a uniform diameter in a range of 0.5 microns to 3 microns at a bottom surface. In some cases, the microwells have a uniform depth in a range of 30 microns to 100 microns. In some cases, the microwells have a largest lateral dimension in a range of 1 to 6 times that of the largest lateral dimension of a cell and/or a bead. In some cases, the microwells have a largest lateral dimension in a range of 1 to 6 times the largest lateral dimension of a cell. In some cases, the microwells have a largest lateral dimension in a range of 1 to 6 times the largest lateral dimension of a bead. In some cases, a total lateral area of microwells at the top surface of the microwell array can comprise at least 10% of the total lateral area of the array. In some cases, the microwells have a uniform diameter in a range of 1 micron to 10 microns. In some cases, the microwells have a uniform diameter in a range of 15 microns to 100 microns. In some cases, each of the microwells can comprise one or more cells.

In some embodiments, the microwell array comprises spatial barcodes. The spatial barcodes can be located inside the microwells such as on an interior surface of the microwells or on a bead that is resident in the microwells. In some embodiments, each of the spatial barcodes is unique. In some embodiments, the array comprises unique spatial barcodes that are unique to each of the microwells or to each cluster of microwells. In some embodiments, the location of each spatial barcode in the microwell array is known. In some embodiments, the spatial barcodes are located at the bottom surfaces of the microwells.

The interior surface of the microwells can be functionalized. In some embodiments, each microwell comprises a functionalized surface that comprises one or more nucleic acid molecules having a unique spatial barcode. In some embodiments, each unique spatial barcode is unique to one or a cluster of wells. In some embodiments, each well contains a unique combination of spatial barcodes. In some embodiments, each unique spatial barcode is co-delivered with a unique stimulus. In some embodiments, the location of each spatial barcode on the array of wells is known.

The microwell array can comprise one or more cut-outs. The one or more cut-outs can be used to direct pipetting. The one or more cut-outs can be independently located anywhere on the array. In some cases, the one or more cut-outs comprise a cut-out located at the center of an array. In some cases, the one or more cut-outs comprise a cut-out located on the side of an array. In some cases, the one or more cut-outs comprise a cut-out located at the center of an array and a cut-out located on the side of an array.

The top surface of the microwell array can be functionalized. In some embodiments, the top surface of the microwell array comprises one or more functional groups such as reactive functional groups. In some embodiments, the reactive functional groups comprise an amine, an aminosilane, a thiosilane, a methacrylate silane, a poly(allylamine), poly(lysine), BSA, epoxide silane, chitosan, 2-iminothiolane, a functional group derived from polyacrylic acid, bisepoexy-PEG, or oxidized agarose, or a combination thereof. The microwell array can comprise glass or a polymer material, for example, poly-dimethylsiloxane (PDMS), polycarbonate (PC), polystyrene (PS), polymethyl-methacrylate (PMMA), PVDF, polyvinylchloride (PVC), polypropylene (PP), cyclic olefin co-polymer (COC), and silicon. In some embodiments, the top surface of the array comprises functional groups conjugated to cyclic olefin co-polymer using aryl diazonium salts. In some embodiments, the top surface of the array bears a charge. In some embodiments, the top surface of the array bears a charge that is opposite to the charge bore on the membrane bottom surface.

In some embodiments, the microwell array used in the present disclosure is a device or system that is suitable for single-cell analysis (e.g., asynchronous single-cell analysis), for example, the devices, systems and methods disclosed in PCT/US20/36197. General description of systems and methods of single cell analyses are described in US2019/0218607A1, which is hereby incorporated in its entirety.

### Cells and Beads

The microwell array can comprise a plurality of beads such as capture beads. In some cases, one or more microwells of the array comprise a single bead. In some cases, at least 80%, 85%, 90%, 95%, 99%, 99.9%, or 100% of microwells in the array comprise a single bead. In some embodiments, less than 10%, 5%, 4%, 3%, 2%, or 1% of the microwells comprise two or more beads. In some cases, beads are pre-loaded into the microwells. In some cases, beads are loaded into the microwells before or after the bioparticles are loaded. In some embodiments, beads and bioparticles are loaded simultaneously. The microwell array can be configured to hold one or more beads. In some embodiments, each of the microwells is configured to hold a single bead. The semi-permeable membrane can be configured to retain the beads such that the beads cannot pass through the membrane pores. The size of the capture beads can be dictated by the size of the microwells that are used. In some embodiments, the size of the bead will be chosen such that only one bead can occupy a microwell at a single time. Alternatively, the dimensions of the microwells can be chosen such that only one bead occupies a microwell at a single time. In some embodiments, the capture beads have an average diameter that is about 1 μm, about 5 μm, about 10 μm, about 15 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 90 μm, about 100 μm, about 110 μm, about 120 μm, about 150 μm, or about 200 μm. In some embodiments, the beads are from about 10 μm-50 μm in diameter. In some embodiments, the beads are about 35 microns in diameter. In some embodiments, the beads are magnetic.

As described herein, a capture bead can comprise a bead having a capture oligonucleotide attached to its surface, which comprises a capture domain, site or sequence for annealing to target nucleic acids such as target transcripts. When the target nucleic acids are transcripts then the bead can be referred to as a “transcript-capture bead”. In some embodiments, the transcript capture bead has a poly(dT) capture sequence for annealing to the poly(dA) tail of mRNA transcripts. In some embodiments, the capture oligonucleotide further comprises a barcode. The barcode can be used for labeling captured nucleic acids from a single cell, including all or a portion of captured transcripts of a single cell. In some embodiments, transcripts of a single cell are captured when the transcript capture bead and the single cell are placed in the same microwell and the cell is lysed. The barcode can be used to label nucleic acids from a single cell or a single microwell. The barcode can also be used to label nucleic acids from a plurality of cells or a plurality of microwells. In some embodiments, a barcode identifies a nucleic acid or a set of nucleic acids as being associated with a particular spatial location and/or with a particular treatment. In some embodiments, a barcode identifies a nucleic acid or a set of nucleic acids as being associated with exposure to a particular stimulus. In some embodiments, a barcode comprises 3, 4, 5, 6, 7, 8, 9, 10, 11, 12, 13, 14, 15, 16, 17, 18, 19, 20, 21, 22, 23 24, 25, 26, 27, 28, 29, or 30 nucleotides. In some embodiments, a barcode comprises 3, 4, 5, 6, 7, 8, 9, 10, 11, 12, 13, 14, 15, or 16 nucleotides. In some embodiments, the capture sequence comprises about 5, 6, 7, 8, 9, 10, 11, 12, 13, 14, 15, 16, 17, 18, 19, 20, 25, or 30 nucleotides. In some embodiments, the capture oligonucleotide comprises about 10, 20, 30, 40, or 50 nucleotides.

The microwell array can comprise one or more bioparticles. In some cases, one or more microwells of the array comprise a single bioparticle (e.g., a single cell). In some cases, at least 80%, 85%, 90%, 95%, 99%, 99.9%, or 100% of microwells in the array comprise a single bioparticle. In some embodiments, less than 10%, 5%, 4%, 3%, 2%, or 1% of the microwells comprise two or more bioparticles. In some specific embodiments, less than 2%, 1.5%, 1%, 0.5%, or 0.1% of the microwells comprise two or more bioparticles. The microwell array can be configured to hold one or more bioparticles. In some embodiments, each of the microwells is configured to hold a single bioparticle. The semi-permeable membrane can be configured to retain the bioparticles such that the bioparticles cannot pass through the membrane pores.

A bioparticle can refer to a particle that comprises biological materials. For example, a bioparticle can refer to a cell or a capture bead that has an RNA attached to it. The one or more bioparticles can comprise a cell, a genome, a nucleic acid, a virus, a nucleus, a protein, or a peptide. In some cases, the bioparticles comprise one or more cells. In some cases, the one or more cells comprise a bacteria cell, a plant cell, an animal cell, or a combination thereof. In some cases, the one or more cells comprise a mammalian cell. In some embodiments, the cells are bacterial cells. In some embodiments, the cells are eukaryotic cells. In some embodiments, the cells are prokaryotic cells. In some embodiments, the cells are murine cells. In some embodiments, the cells are primate cells. In some embodiments, the cells are human cells. In some embodiments, the cells are tumor cells. The cells (or nucleic acid source) can be naturally occurring or it can be non-naturally occurring. In some embodiments, the cells are healthy cells. In some embodiments, the cells are diseased cells.

In some embodiments, the cells are mammalian cells. The mammalian cells can comprise one or more blood cells such as white blood cell (e.g., monocytes, lymphocytes, neutrophils, eosinophils, basophils, and macrophages), red blood cell (erythrocytes), or platelet.

In some embodiments, the method comprises loading a sample fluid. In some embodiments, the method comprises contacting the microwell array with a sample fluid. In some embodiments, the method comprises contacting the microwell array with a tissue sample. The sample fluid can be loaded manually or by automation. In some embodiments, the sample fluid is loaded by pipetting. In some embodiments, the sample fluid is loaded by flowing a sample solution over the loading assembly. The loading of the sample fluid can be directed by the one or more cut-outs in the array, the opening(s) in the lid, or both. In some embodiments, the sample fluid is loaded to the cut-out area in the array. A suitable volume of the loaded sample fluid can depend on various factors, including but not limited to, the size of the array, the number and volume of the microwells in the array, the concentration of the sample fluid, etc. In some embodiments, the sample fluid comprises from about 0.1 mL to about 5 mL liquid. In some specific embodiments, the sample fluid comprises about 0.2 mL, about 0.3 mL, about 0.4 mL, about 0.5 mL, about 0.6 mL, about 0.7 ml, about 0.8 mL, about 0.9 mL, about 1.0 mL, about 1.1 ml, about 1.2 mL, about 1.3 mL, about 1.4 mL, about 1.5 mL, about 1.6 mL, about 1.7 mL, about 1.8 mL, about 1.9 mL, or about 2.0 mL of fluid.

The sample fluid can comprise one or more bioparticles. In some embodiments, the sample fluid comprises a plurality of bioparticles. The bioparticles can exist in the sample fluid in various forms; for example, the bioparticles can be dissolved in the sample fluid, suspended in the sample fluid, or in micelles that are distributed in the sample fluid. In some specific embodiments, the sample fluid comprises a suspension of cells.

In some embodiments, the ratio of the number of bioparticles in the sample fluid to the number of microwells in the microwell array can be from about 1:1000 to about 10:1. In some cases, the ratio of the number of bioparticles in the sample fluid to the number of microwells in the microwell array can be from about 1:100 to about 1:1, from about 1:20 to about 1:4, or from about 1:10 to about 1:8. In some cases, the ratio of the number of bioparticles in the sample fluid to the number of microwells in the microwell array is from about 1:10 to about 1:8. In some embodiments, at least 50%, at least 60%, at least 70%, at least 80%, at least 90%, at least 91%, at least 92%, at least 93%, at least 94%, at least 95%, at least 96%, at least 97%, at least 98%, or at least 99% of bioparticles in the sample fluid are loaded in microwells. In some embodiments, at least 95% of bioparticles in the sample fluid are loaded in microwells.

After the sample fluid is loaded, one or more of the microwells can comprise one or bioparticles. In some embodiments, at least 0.5%, at least 1%, at least 2%, at least 5%, at least 10%, at least 15%, at least 20%, at least 30%, or at least 50% of the microwells comprise one or more bioparticles. In some embodiments, at least 0.5%, at least 1%, at least 2%, at least 5%, at least 10%, at least 15%, at least 20%, at least 30%, or at least 50% of the microwells comprise a single bioparticle. In some embodiments, from about 5% to about 20%, from about 5% to about 15%, or from about 8% to about 12% of the microwells comprise a single bioparticle. In some embodiments, the rest of the microwells are not occupied by any bioparticles. In some embodiments, less than 10%, less than 5%, less than 2%, or less than 1% of the microwells comprise two or more bioparticles.

In some embodiments, the method comprises mixing the loaded sample fluid. The mixing can be provided by agitating the loaded sample fluid, e.g., by pipetting one or more times. The mixing can be provided by swirling the loading assembly after the sample has been loaded. The mixing can also be provided by tilting the loading assembly. In some embodiments, the mixing comprises one or more means, such as agitating and swirling. In some specific embodiments, the method comprises agitating the loaded fluid by pipetting one or more times (such as 1-10 times). In some embodiments, the sample fluid is agitated at a cut-out at the center of the array.

In some cases, the method can further comprise incubating a loaded sample fluid. The sample fluid can be incubated before the mixing, after the mixing, or both. In some embodiments, the sample fluid is incubated statically before the mixing (e.g., agitation). In some embodiments, the sample fluid is incubated statically after the mixing. The sample fluid can be incubated for a period of time. In some embodiments, the incubation time is from about 30 seconds to about 12 hours, from about 1 minute to about 1 hour, or from about 2 minutes to about 15 minutes, for each incubation. In some embodiments, the incubation time is from about 1 minute to about 10 minutes or from about 3 minutes to about 7 minutes. In some embodiments, the incubation time is about 5 minutes.

The method can comprise preserving the bioparticles after the sample fluid has been loaded. In some embodiments, the method comprises counting target nucleic acid molecules (e.g., RNA) in a preserved bioparticle. In some embodiments, the method comprises applying a storage buffer to the microwell array after a sample fluid is loaded. The storage buffer can operate to preserve the bioparticles or one or more biomaterials within the bioparticles. In some embodiments, the storage buffer operates to preserve polynucleic acids such as RNAs in the cells. The method can further comprise incubating the bioparticles in the presence of a storage buffer. In some cases, the method can comprise removing the loading ring after a sample fluid is loaded. In some cases, the loading ring is removed after the storage buffer has been loaded.

The methods can comprise storing at least one retained bioparticle for one or more days and counting target nucleic acid molecules (e.g., RNA) of the bioparticle. The microwell array that comprises the bioparticle can also be placed into long term storage at a temperature below 0° C., including for example at about −80° C. or at about −20° C. In some embodiments, the microwell array that comprises one or more bioparticles is stored for a period of time that is between 1 hour and 30 years. For example, the microwell array can be stored for at least 1 day, at least 1 week, at least a month, or at least a year. For example, the microwell array can be stored for at most 1 day, at most 1 week, at most a month, at most a year, or at most 30 years. The method can further comprise shipping the microwell array that comprises one or more bioparticles. In some embodiments, the microwell array is shipped from a point of care facility such as a clinic to a central processing and/or analytical center.

The method can comprise means of exposing the backside of the membrane (i.e., membrane top surface). After the membrane top surface is exposed, bioparticles retained in the microwells can be further processed. In some embodiments, such processing comprises lysing the cells retained in the microwells. In some embodiments, the method comprises contacting one or more lysis buffers with the array. The method can comprise lysing at least one cell, thereby releasing an RNA from the cell. The released RNA can then be captured by a capture bead that is resident in the same microwell as the lysed cell. Accordingly, in some embodiments, the method comprises capturing RNA on a bead resident in the same microwell as at least one cell. In some embodiments, other biomaterials released by the cell such as a DNA, an antibody, or a protein is captured by the capture bead.

The beads can be pre-loaded into the microwells. In some cases, a microwell array can be pre-loaded with a plurality of beads. In some embodiments, the beads are pre-loaded in a dry state. Alternatively, the beads can be loaded before, after, or simultaneously as the sample fluid. In some embodiments, at least 80%, at least 90%, at least 91%, at least 92%, at least 93%, at least 94%, at least 95%, at least 96%, at least 97%, at least 98%, at least 99%, or at least 99.9% of the microwells are loaded with a single bead. In some cases, the beads are barcoded transcript capture beads. In some embodiment, depending on the application, one or more stimuli can be added to the microwells.

The method can comprise aggregating the one or more bioparticles in the microwells. In some cases, the method comprises collecting at least a portion of the bioparticles. In some cases, the method comprises collecting at least a portion of the plurality of beads. In some cases, a method can further comprise generating cDNA from a captured RNA such that a sequence of a bead barcode can be incorporated into a cDNA. In some embodiments, the method comprises counting template nucleic acid molecules (e.g., cDNA) in a bioparticle, thereby counting the target nucleic acid molecules therein.

In some cases, automation can be used to perform these methods. It will be appreciated that the same approach can be adopted for other nucleic acid sources that can be analyzed using the methods and products of this disclosure including without limitation viruses, nuclei, exosomes, platelets, etc.

### Computer Systems

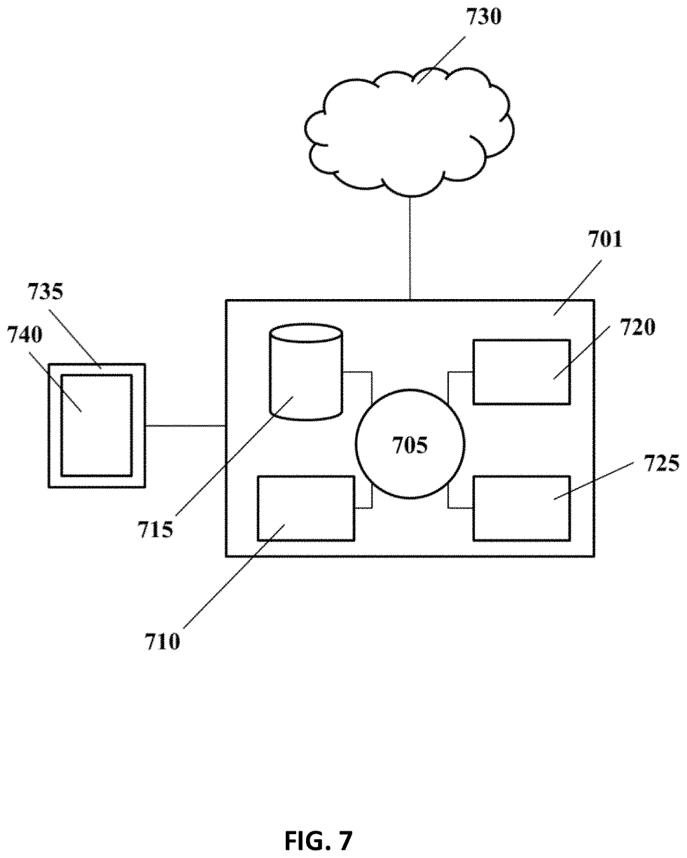

The present disclosure provides computer systems that are programmed to implement methods of the disclosure. FIG. 7 shows a computer system 701 that is programmed or otherwise configured to, for example, sequence nucleic acid molecules to produce sequencing reads, align sequencing reads to a reference sequence, determine a number of unique truncation base positions present in amplified nucleic acid molecules, and identify a number of nucleic acid molecules present in a sample.

The computer system 701 can regulate various aspects of analysis, calculation, and generation of the present disclosure, such as, for example, sequencing nucleic acid molecules to produce sequencing reads, aligning sequencing reads to a reference sequence, determining a number of unique truncation base positions present in amplified nucleic acid molecules, and identifying a number of nucleic acid molecules present in a sample. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet.

The network 730 in some cases is a telecommunication and/or data network. The network 730 can include one or more computer servers, which can enable distributed computing, such as cloud computing. For example, one or more computer servers can enable cloud computing over the network 730 (“the cloud”) to perform various aspects of analysis, calculation, and generation of the present disclosure, such as, for example, sequencing nucleic acid molecules to produce sequencing reads, aligning sequencing reads to a reference sequence, determining a number of unique truncation base positions present in amplified nucleic acid molecules, and identifying a number of nucleic acid molecules present in a sample. Such cloud computing can be provided by cloud computing platforms such as, for example, Amazon Web Services (AWS), Microsoft Azure, Google Cloud Platform, and IBM cloud. The network 730, in some cases with the aid of the computer system 701, can implement a peer-to-peer network, which can enable devices coupled to the computer system 701 to behave as a client or a server.

The CPU 705 can comprise one or more computer processors and/or one or more graphics processing units (GPUs). The CPU 705 can execute a sequence of machine-readable instructions, which can be embodied in a program or software. The instructions can be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 701 can include or be in communication with an electronic display 735 that comprises a user interface (UI) 740 for providing, for example, a visual display indicative of sequencing reads, sequencing reads aligned to a reference sequence, a number of unique truncation base positions determined to be present in amplified nucleic acid molecules, and a number of nucleic acid molecules identified to be present in a sample. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, sequence nucleic acid molecules to produce sequencing reads, align sequencing reads to a reference sequence, determine a number of unique truncation base positions present in amplified nucleic acid molecules, and identify a number of nucleic acid molecules present in a sample.

### EXAMPLES

### Example 1—Counting Genes and Transcripts Using Truncation Mapping Sites

Peripheral blood mononuclear cells (PBMCs) were loaded into a microwell array that was configured for single cell analysis, such that one or more microwells in the array were loaded with a single cell. The microwell array was preloaded with a single barcoded bead in a plurality of the microwells. Each of the barcoded bead contained multiple barcodes that each comprised a first strand synthesis primer.

The first strand synthesis primer contained, in 5′ to 3′ direction, a 5′ universal primer sequence, a sided sequence (3′SS), a cell barcode, and poly(dT) sequence. It has a sequence of AAGCAGTGGTATCAACGCAGAGTACJJJJJJJJJJJJTTTTTTTTTTTTTTTTTTTTTTTTTTTTT T, where J is the random cell barcode generated through split and pool synthesis. The cell barcode was the same for each bead, and thus associating each first strand cDNA molecule and any subsequent products of the cDNA with the single cell to which the bead was associated with.

The array was sealed with a semipermeable membrane and then submerged in a 5 molar (M) guanidine thiocyanate (GITC) buffer for 15 minutes, followed by 30 minutes in a 2 M sodium chloride (NaCl) solution. The mRNAs in the samples were released and attached to the bead through the poly(dT). The membrane was then removed, and the beads, to which a plurality of mRNAs from the cells were attached, were recovered by centrifugation. Reverse transcription was performed for 1 hour at 37° C. to convert captured mRNA molecules into first strand cDNA molecules. The beads were then washed with 0.1 M sodium hydroxide (NaOH) for 5 minutes to denature the cDNA hybrid molecules.

The resultant cDNA molecules, which are attached to the beads, contained the 5′ universal primer sequence, the sided sequence (3′SS), the cell barcode, the poly(dT) sequence, and a copy of an mRNA sequence.

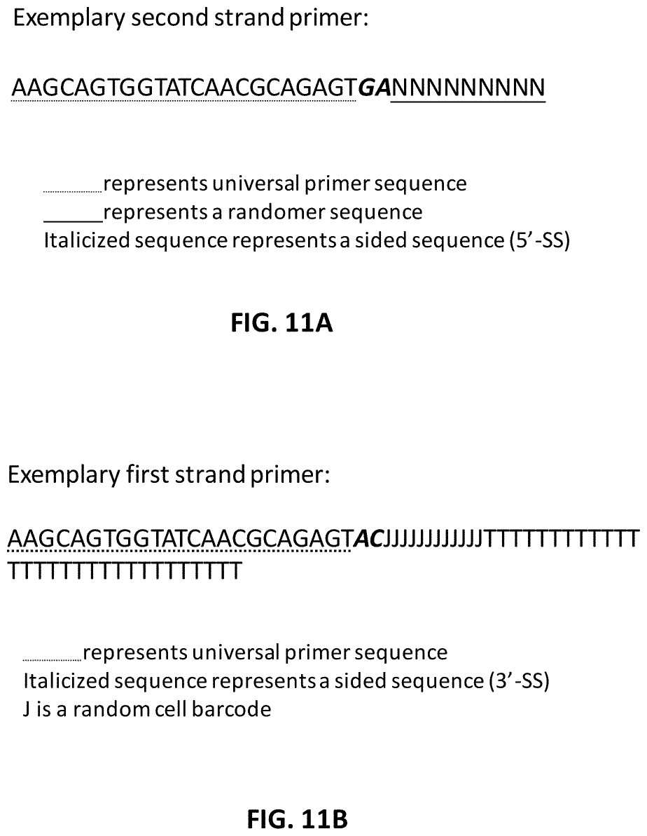

Next, second strand synthesis was performed by incubating the beads and a second strand primer (AAGCAGTGGTATCAACGCAGAGTGANNNNNNNNN) (see, FIG. 11A) with 25 U of Klenow exo—in 200 μL of 50 mM Tris pH 8.3, 75 mM potassium chloride (KCl), 12% PEG8000, 1 mM deoxynucleoside triphosphates (dNTPs), 3 mM magnesium chloride (MgCl₂), and 10 mM Dithiothreitol (DTT) for 30 minutes at 37° C. N is any nucleotide. The second strand products (i.e., second strand cDNA molecules) were amplified by polymerase chain reaction (PCR) using Kapa HiFi and primer (AAGCAGTGGTATCAACGCAGAGT). The whole transcriptome amplification (WTA) product was purified by SPRI purification.

The unique truncation sites were created when the second strand primers randomly attach to a position on the first strand cDNAs. The unique truncation sites were preserved in the second strand cDNA molecules and in the amplified products.

A portion of the WTA product was directly indexed for sequencing in a second PCR reaction using Kapa HiFi and two indexing primers—CAAGCAGAAGACGGCATACGAGATTCGCCTTAGAGACATACACCCTCGTCGGACATCA ACGCAGAGT*G*A and AATGATACGGCGACCACCGAGATCTACACTATCCTCTCGCCCAGGAAGACACCGGTAC AATCAACGCAGAGT*A*C. “*” represents a phosphorothioate bond. The amplification program was run at 98° C. for 3 min, followed by 15 cycles each of 98° C. for 30 seconds, 60° C. for 5 minutes, and 72° C. for 30 seconds. Other indexing primers can also be used.

The product was purified and sequenced on an Illumina NextSeq sequencer with the following sequencing primers—Read1—CGC CCA GGA AGA CAC CGG TAC AAT CAA CGC AGA GTA C and Read2—GAG ACA TAC ACC CTC GTC GGA CAT CAA CGC AGA GTG A.

Next, each sequencing run was aligned to the human genome using default settings of the STAR aligner tool to identify reads mapping to exons. The cell barcode was extracted from read1 on each molecule. The mapping location of each read was also extracted. Reads with the same cell barcode were aggregated. Transcripts with the same mapping base+/−1 base were collapsed into a single transcript count.

### Example 2—Comparative Example: Counting Genes and Transcripts Using Unique Molecular Indices (UMIs) or Truncation Mapping Sites

Peripheral blood mononuclear cells (PBMCs) were loaded into a microwell array that was configured for single cell analysis, such that one or more microwells in the array were loaded with a single cell. The microwell array was preloaded with barcoded poly(dT) capture beads, which contained first strand synthesis primers that each comprising a cell barcode (i.e., sample barcode) that is common to each bead, a unique molecular identifiers (UMIs), and a universal primer sequence. The array was sealed with a semipermeable membrane and then submerged in a 5 molar (M) guanidine thiocyanate (GITC) buffer for 15 minutes, followed by 30 minutes in a 2 M sodium chloride (NaCl) solution. The membrane was then removed, and the beads, to which a plurality of mRNAs from the cells were attached, were recovered by centrifugation. Reverse transcription was performed for 1 hour at 37° C. to convert captured mRNA molecules into first strand cDNA molecules. The beads were then washed with 0.1 M sodium hydroxide (NaOH) for 5 minutes to denature the cDNA hybrid molecules.

Next, second strand synthesis was performed by incubating the beads and a second strand primer (AAGCAGTGGTATCAACGCAGAGTGANNNNNNNNN) (see, FIG. 11A) with 25 U of Klenow exo—in 200 μL of 50 mM Tris pH 8.3, 75 mM potassium chloride (KCl), 12% PEG8000, 1 mM deoxynucleoside triphosphates (dNTPs), 3 mM magnesium chloride (MgCl₂), and 10 mM Dithiothreitol (DTT) for 30 minutes at 37° C. The second strand products (i.e., second strand cDNA molecules) were amplified by polymerase chain reaction (PCR) using Kapa HiFi and primer (AAGCAGTGGTATCAACGCAGAGT). The whole transcriptome amplification (WTA) product was purified by SPRI purification. The unique truncation sites were created when the second strand primers randomly attach to a position on the first strand cDNAs, and the unique truncation sites were preserved in the second strand cDNA molecules and in the amplified products.

In truncation mapping site method, a portion of the WTA product was directly indexed for sequencing in a second PCR reaction using Kapa HiFi and two indexing primers—CAAGCAGAAGACGGCATACGAGATTCGCCTTAGAGACATACACCCTCGTCGGACATCA ACGCAGAGT*G*A and AATGATACGGCGACCACCGAGATCTACACTATCCTCTCGCCCAGGAAGACACCGGTAC AATCAACGCAGAGT*A*C. The amplification program was run at 98° C. for 3 min, followed by 15 cycles each of 98° C. for 30 seconds, 60° C. for 5 minutes, and 72° C. for 30 seconds.

The product was purified and sequenced on an Illumina NextSeq sequencer with the following sequencing primers—Read1—CGC CCA GGA AGA CAC CGG TAC AAT CAA CGC AGA GTA C and Read2—GAG ACA TAC ACC CTC GTC GGA CAT CAA CGC AGA GTG A.

Separately, in UMI method, a second portion of the initial amplification product was subjected to tagmentation using Illumina Nextera XT. The tagmented library was amplified with standard primers, purified and sequenced on an Illumina NextSeq with the following primers—Read1—GCCTGTCCGCGGAAGCAGTGGTATCAACGCAGAGTAC and Read2—Nextera read 2 sequencing primer. The unique truncation mapping sites were not preserved in the tagmented library.

Next, each sequencing run was aligned to the human genome using default settings of the STAR aligner tool to identify reads mapping to exons. The cell barcode was extracted from read1 as was the UMI sequence on each molecule. The mapping location of each read was also extracted. Reads with the same cell barcode were aggregated. Transcript counts from the library generated without tagmentation were acquired by either collapsing all reads mapping to the same transcript with an identical UMI or a UMI which was 1 Hamming distance away in sequencing space (e.g., identical except for a difference in a single base). Alternatively, transcripts with the same mapping base+/−1 base were collapsed into a single transcript count.

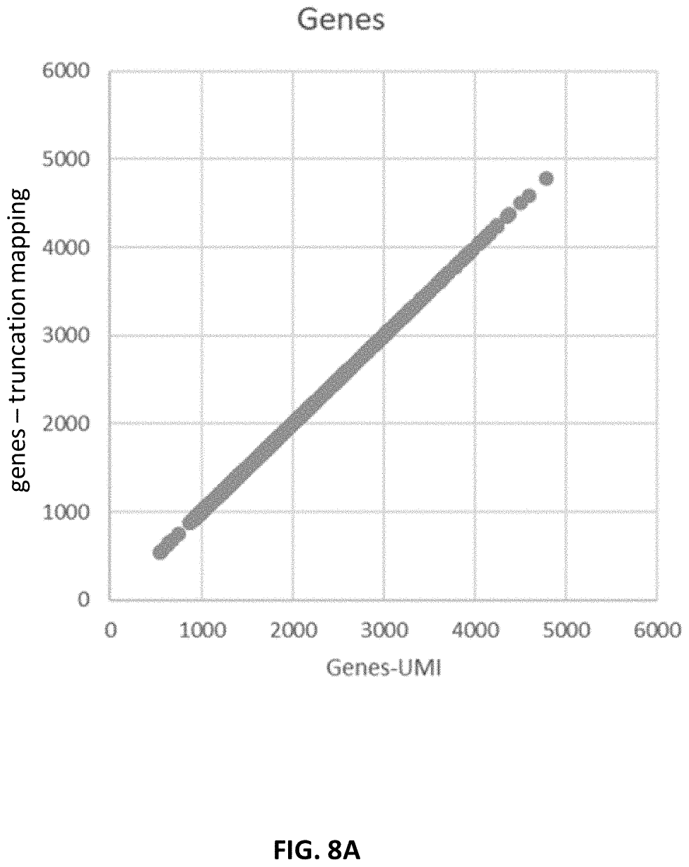

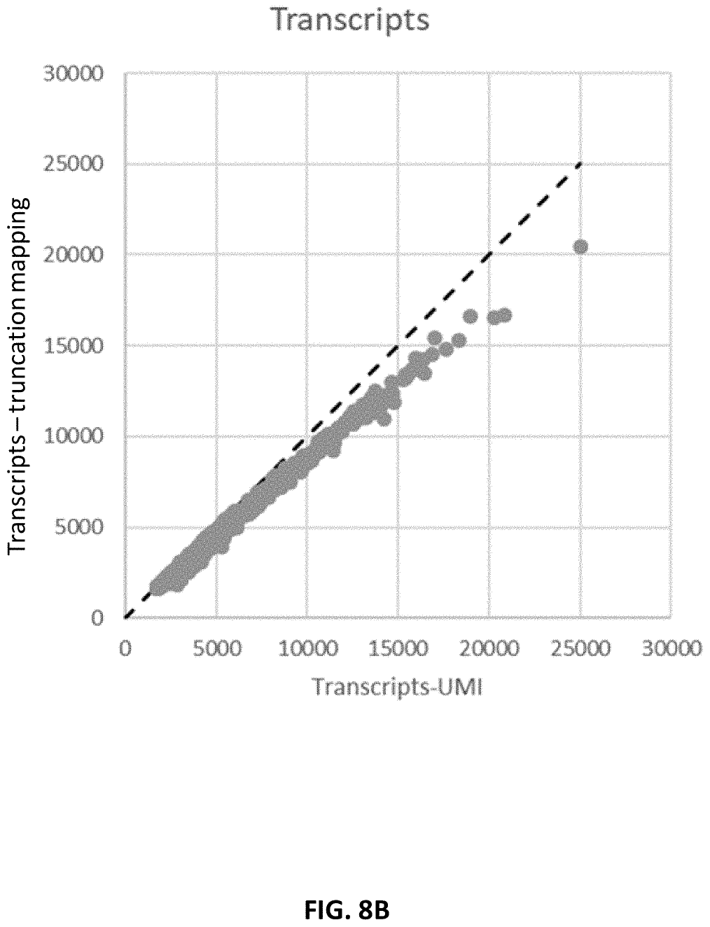

FIGS. 8A and 8B show an example comparison of gene and transcript counting, respectively, using unique molecular indices or truncation mapping site on same sequencing data, in accordance with disclosed embodiments. The total gene count (FIG. 8A) and transcript count (FIG. 8B), as determined by mapping location, are plotted as a function of the number of gene or transcript counts determined for the same cell using UMI tags. FIG. 8A shows that the total gene counts were substantial the same when determined based on unique molecular indices or truncation mapping site. FIG. 8B shows that the transcript counts were substantial the same when determined based on unique molecular indices or truncation mapping, particularly for transcript counts under 15,000.

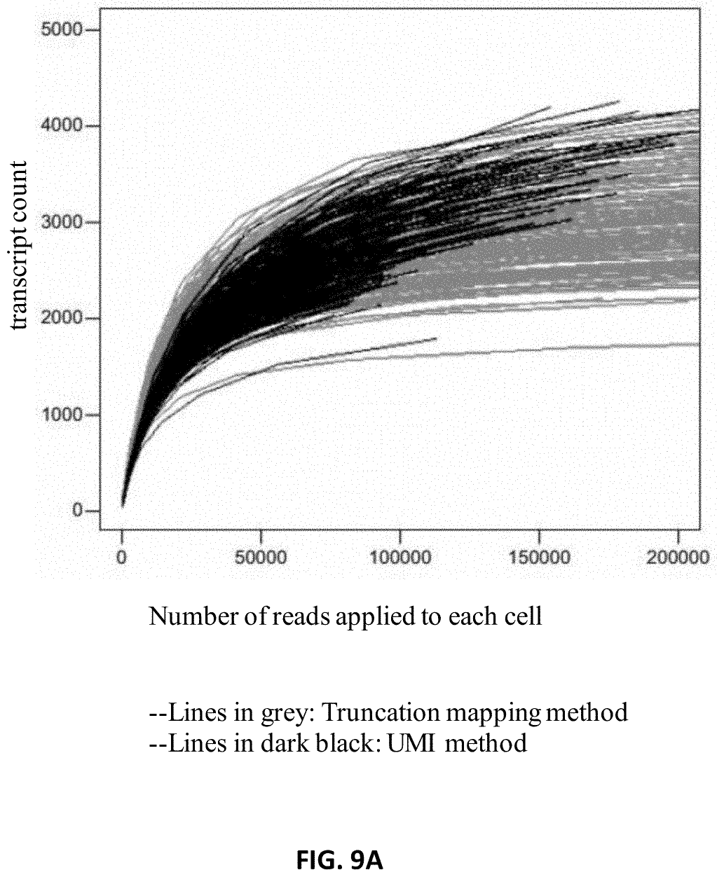

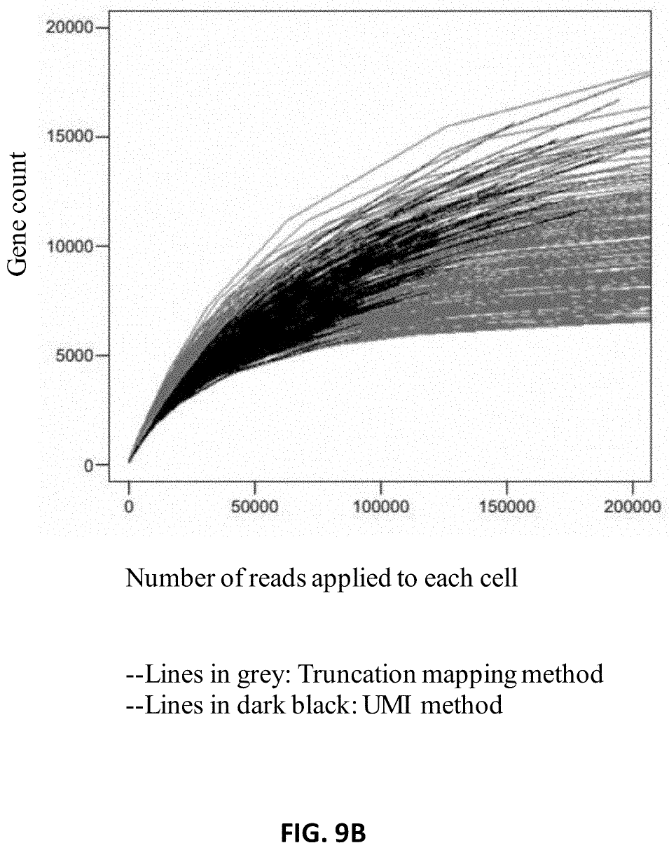

FIGS. 9A and 9B show example plots of gene and transcript yields per cell, respectively, as a function of sequencing read depth from libraries generated with the standard second strand synthesis protocol or the truncated protocol, in accordance with disclosed embodiments. The complexity of the libraries produced by the direct indexing PCR or tagmentation is illustrated. The total transcript count (FIG. 9A) and gene count (FIG. 9B), are plotted as a function of the number of reads applied to each cell. This is determined by downsampling the sequencing reads, and re-calculating the transcript and gene counts. Each trace is a measure of the saturation of a single cell transcriptome as more sequencing reads are applied. The grey lines represents transcripts or genes determined by truncation mapping method, and the dark black lines represents transcripts or genes determined by UMI methods. As illustrated in FIGS. 9A-9B, both methods provide similar gene and transcripts yield per single cell.

### Example 3—Counting Genes and Transcripts Using Truncation Mapping Sites

PBMC were loaded into a nanowell array with barcoded transcript capture beads. The capture beads comprised first strand synthesis primers attached thereto. The first strand synthesis primers were configured according to FIG. 4. The array was sealed with a semi-permeable membrane. Cells were lysed and released RNA was captured on the beads. After beads were recovered from the array, whole transcriptome amplification was achieved through reverse transcription, exonuclease digestion of un-extended probes, randomly-primed second strand synthesis with tailed poly(N) primers and PCR amplification using a universal primer. The second strand synthesis primers used in the second strand synthesis were configured according to FIG. 4. The sequencing adaptors were added to the appropriate sides through a second PCR reaction that used primers specific for the 5′ and 3′ sided sequence with 5′ tails containing the appropriate adaptor. The library was sequenced with the cellular barcode being captured in read 1 and the truncation mapping site and transcript identity being captured in read 2. During bioinformatic analysis, molecule counting was calculated by counting the number of unique truncation mapping sites for each gene for each cell.

### Example 4—Comparative Example: Counting Genes and Transcripts Using Truncation Mapping Sites

PBMC were loaded into a nanowell array with barcoded transcript capture beads. The capture beads comprised first strand synthesis primers attached thereto. The first strand synthesis primers were configured according to FIG. 4. The array was sealed with a semi-permeable membrane. Cells were lysed and released RNA was captured on the beads. After beads were recovered from the array, whole transcriptome amplification was achieved through reverse transcription, exonuclease digestion of un-extended probes, randomly-primed second strand synthesis with tailed poly(N) primers and PCR amplification using a universal primer. The second strand synthesis primers used in the second strand synthesis were configured according to FIG. 4. The sequencing adaptors were added to the appropriate sides through a second PCR reaction that used primers specific for the 5′ and 3′ sided sequence with 5′ tails containing the appropriate adaptor. The library was sequenced with the cellular barcode and UMI sequence being captured in read 1 and the truncation mapping site and transcript identity being captured in read 2. During bioinformatic analysis, molecule counting was calculated by counting the unique number of UMIs associated with each gene for each cell or the number of unique truncation mapping sites for each gene for each cell.

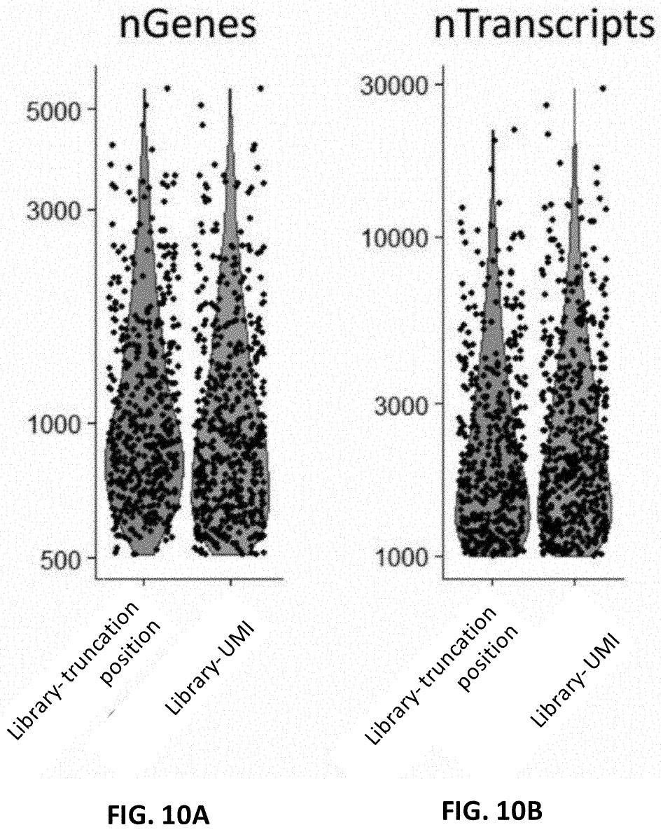

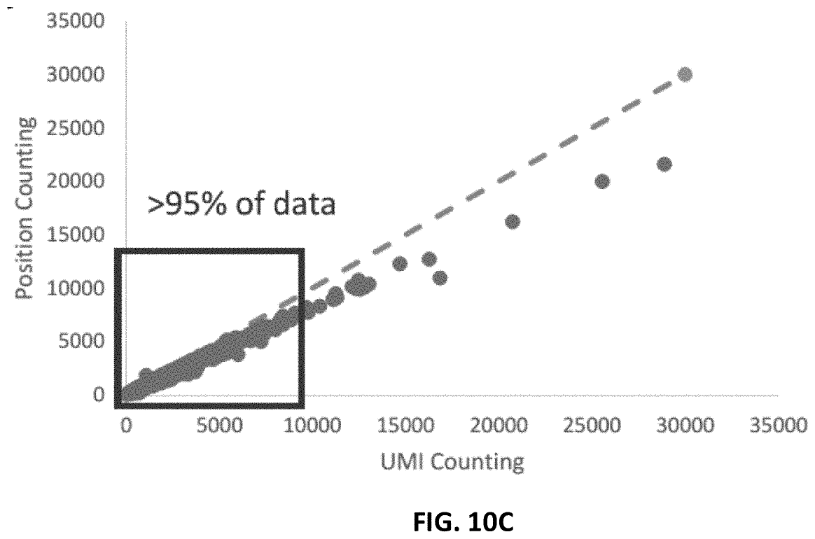

FIGS. 10A and 10B show the gene and transcript per cell yields respectively from single cell libraries employing unique molecular identifiers or truncation site as the molecule counter. FIG. 10C displays the transcript count as determined by UMI analysis for each cellular barcode as a function of the transcript count from the same barcodes as determined by truncation mapping. A perfect 1:1 match is plotted as a dashed line. FIG. 10C shows that >95% of the cellular transcriptomes lie within an area where the UMI and truncation mapping methods are very close to the theoretical 1:1 match line, indicating very similar transcript counts.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein can be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for counting nucleic acid molecules of a sample, comprising:
(a) obtaining a sample comprising a plurality of template nucleic acid molecules;
(b) randomly truncating said plurality of template nucleic acid molecules at a truncation base position within said plurality of template nucleic acid molecules, wherein said truncating comprises performing a random selection of said truncation base position among a plurality of base positions of said template nucleic acid molecule, thereby producing a plurality of truncated nucleic acid molecules,
wherein said plurality of template nucleic acid molecules comprises cDNA molecules,
wherein said truncating comprises making a copy of at least a portion of said plurality of template nucleic acid molecules, and forming a plurality of second strand cDNA molecules from said plurality of template nucleic acid molecules, wherein said truncation base positions are preserved in said plurality of second strand cDNA molecules;
(c) amplifying at least a portion of said plurality of truncated nucleic acid molecules to produce a plurality of amplified nucleic acid molecules, wherein said truncation base positions are preserved in said amplified nucleic acid molecules;
(d) sequencing at least a portion of said plurality of amplified nucleic acid molecules to produce a plurality of sequencing reads, wherein each of said plurality of sequencing reads comprises a truncation location corresponding to said truncation base position of said corresponding amplified nucleic acid molecule;
(e) aligning at least a portion of said plurality of sequencing reads to a reference sequence, thereby producing a plurality of aligned sequencing reads; and
(f) identifying a number of template nucleic acid molecules present in said sample using truncation locations of said plurality of aligned sequencing reads.

2-6. (canceled)

7. The method of claim 1, further comprising processing at least a portion of said amplified nucleic acid molecules to produce a sequencing library, wherein said truncation base positions are preserved in said sequencing library.

8. (canceled)

9. (canceled)

10. The method of claim 1, wherein said sample comprises one or more barcoded beads, and wherein said template nucleic acid molecules are cDNA molecules attached to said barcoded beads, and wherein said cDNA molecules are obtained by reverse transcription of RNA molecules that are released from cellular single cell samples.

11. (canceled)

12. (canceled)

13. (canceled)

14. The method of claim 1, further comprising contacting said plurality of template nucleic acid molecules with a plurality of second strand primers, wherein each of said plurality of second strand primers comprises a 5′ universal primer sequence and a 3′ sequence complementary to a sequence of said template nucleic acid molecules, and wherein said 3′ sequence comprises a random sequence, and further comprising extending said plurality of second strand primers to produce said plurality of second strand cDNA molecules.

15. (canceled)

16. (canceled)

17. (canceled)

18. (canceled)

19. (canceled)

20. The method of claim 14, wherein said second strand primers comprise a sided sequence (SS), wherein said SS comprises 5 to 9 bases.

21. (canceled)

22. (canceled)

23. The method of claim 14, wherein said template nucleic acid molecules comprise, in 5′ to 3′ direction, a universal primer sequence, a sided sequence (SS), a sample barcode, a poly(dT) sequence, and a sequence that is complementary to a sequence of a target nucleic acid.

24-39. (canceled)

40. The method of claim 7, wherein said method comprises a PCR amplification that re-establishes directionality of said sequencing library.

41. The method of claim 7, wherein said sequencing library comprises known sided sequences (SS) on a 3′ and a 5′ side of nucleic acid molecules of said sequencing library, wherein the 3′ and 5′ SS defines the 3′ and 5′ direction of the sequencing library respectively.

42. The method of claim 41, wherein said 3′ SS is a copy of the SS in the template nucleic acid molecules, and said 5′ SS is a copy of the SS in the second strand primer.

43-70. (canceled)

71. The method of claim 1, wherein said sequencing comprises obtaining a first sequencing read and a second sequencing read, wherein said sample barcode is captured in said first sequencing read and wherein said truncation location corresponding to said truncation base position is captured in said second read.

72-86. (canceled)

87. A method for enriching a sample for one or more target sequences, comprising:
(a) obtaining a sample comprising a plurality of template nucleic acid molecules, wherein said template nucleic acid molecules comprise one or more target sequences;
(b) combining said plurality of template nucleic acid molecules with a set of blocking oligonucleotides, wherein said set of blocking oligonucleotides comprises a sequence complementary to a template nucleic sequence that is 3′ to one of said target sequences, thereby annealing said template nucleic acid sequence that is 3′ to one of said target sequences with at least one of said set of blocking oligonucleotides;
(c) contacting said plurality of template nucleic acid molecules with a plurality of second strand primers, wherein said plurality of second strand primers comprises a 5′ universal primer sequence and a 3′ sequence complementary to a sequence of said template nucleic acid; and
(d) extending said second strand primers to produce a plurality of second strand nucleic acid molecules, thereby enriching at least one of said one or more target sequences.

88. The method of claim 87, further comprising extending said second strand nucleic acid molecules through a region of said second strand cDNA molecule corresponding to a blocking oligonucleotide of said set of blocking oligonucleotides to acquire a 3′ barcode and a 3′ UPS sequence.

89. The method of claim 87, further comprising performing a two-step extension reaction using a mesophilic DNA polymerase and a thermophilic DNA polymerase.

90. (canceled)

91. (canceled)

92. The method of claim 87, further comprising annealing said set of blocking oligonucleotides and said 3′ sequences, and extending said set of blocking oligonucleotides using a DNA polymerase and one or more cleaving enzymes corresponding to said set of blocking oligonucleotides.

93-144. (canceled)

145. A method for counting target mRNA nucleic acid molecules of a single cell sample, comprising:
(a) isolating a single cell sample;
(b) releasing target mRNA nucleic acid molecules from said single cell sample;
(c) capturing said target nucleic acid molecules onto a barcoded bead that is associated with said single cell sample;
(d) making first strand cDNA molecules by performing reverse transcription of said target mRNA nucleic acid molecules, wherein said first strand cDNA molecules each comprises a copy of a sequence of said target mRNA molecules;
(e) randomly truncating said first strand cDNA molecules at a truncation base position within said plurality of first strand cDNA molecules, wherein said truncating comprises randomly attaching a second strand synthesis primer to the first strand cDNA molecules and extending the synthesis primer, thereby producing a plurality of second strand cDNA molecules each preserving the base position at which the second strand synthesis primer is attached;
(f) amplifying at least a portion of said second strand cDNA molecules to produce a plurality of amplified nucleic acid molecules, wherein said truncation base positions are preserved in said amplified nucleic acid molecules;
(g) sequencing at least a portion of said plurality of amplified nucleic acid molecules to produce a plurality of sequencing reads, wherein said truncation base positions are preserved in said plurality of sequencing reads;
(h) aligning at least a portion of said plurality of sequencing reads to a reference sequence, thereby producing a plurality of aligned sequencing reads; and
(i) correlating a number of target mRNA molecules present in said single cell using truncation locations of said plurality of aligned sequencing reads, thereby counting target mRNA nucleic acid molecules.

146. The method of claim 145, wherein the first strand cDNA molecules comprise a universal primer sequence, a sided sequence that is configured to establish directionality, a sample barcode, a poly(dT) sequence, and a sequence that comprises a copy of at least a portion of the target mRNA molecule.

147. The method of claim 145, wherein the first strand cDNA molecules comprise a universal primer sequence, a sided sequence that is configured to establish directionality, a sample barcode, a sequence that is complementary to a sequence of the target mRNA, and a sequence that comprises a copy of at least a portion of the target mRNA molecule.

148. The method of claim 145, wherein the second strand synthesis primer comprise a universal primer sequence, a sided sequence that is configured to establish directionality, and a sequence that is complementary to a sequence of the first strand cDNA molecule.

149. The method of claim 148, wherein the sequence that is complementary to a sequence of the first strand cDNA molecule is a random sequence.

150. The method of claim 148, wherein the sided sequences is 5 to 9 bases in length.

151-154. (canceled)


SEQUENCE LISTING (8 sequences)

SEQ ID NO: 1 - DNA, 67 bp, Artificial Sequence
  Description of Artificial Sequence Synthetic primer
  modified_base (26)..(37): a, c, t, g, unknown or other
aagcagtggtatcaacgcagagtacnnnnnnnnnnnntttttttttttttttttttttttttttttt

SEQ ID NO: 2 - DNA, 34 bp, Artificial Sequence
  Description of Artificial Sequence Synthetic primer
  modified_base (26)..(34): a, c, t, g, unknown or other
aagcagtggtatcaacgcagagtgannnnnnnnn

SEQ ID NO: 3 - DNA, 23 bp, Artificial Sequence
  Description of Artificial Sequence Synthetic primer
aagcagtggtatcaacgcagagt

SEQ ID NO: 4 - DNA, 69 bp, Artificial Sequence
  Description of Artificial Sequence Synthetic primer
caagcagaagacggcatacgagattcgccttagagacatacaccctcgtcggacatcaacgcagagtga

SEQ ID NO: 5 - DNA, 74 bp, Artificial Sequence
  Description of Artificial Sequence Synthetic primer
aatgatacggcgaccaccgagatctacactatcctctcgcccaggaagacaccggtacaatcaacgcagagtac

SEQ ID NO: 6 - DNA, 37 bp, Artificial Sequence
  Description of Artificial Sequence Synthetic primer
cgcccaggaagacaccggtacaatcaacgcagagtac

SEQ ID NO: 7 - DNA, 37 bp, Artificial Sequence
  Description of Artificial Sequence Synthetic primer
gagacatacaccctcgtcggacatcaacgcagagtga

SEQ ID NO: 8 - DNA, 37 bp, Artificial Sequence
  Description of Artificial Sequence Synthetic primer
gcctgtccgcggaagcagtggtatcaacgcagagtac